(19) United States
(12) Patent Application Publication
(10) Pub. No.: US 2004/0137429 A1
(43) Pub. Date: Jul. 15, 2004

(54) Therapeutic aptamers having binding specificity to gp41 of HIV

(76) Inventors: David Epstein, Belmont, MA (US); Jill Blanchard, Arlington, MA (US); Charles Wilson, Concord, MA (US); John L. Diener, Cambridge, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/677,807

(22) Filed: Oct. 1, 2003

Related U.S. Application Data
(60) Provisional application No. 60/415,390, filed on Oct. 2, 2002.
(60) Provisional application No. 60/441,416, filed on Jan. 21, 2003.
(60) Provisional application No. 60/461,966, filed on Apr. 10, 2003.
(60) Provisional application No. 60/465,148, filed on Apr. 23, 2003.

(51) Int. Cl. C12Q 1/70; A61K 39/21; C07K 14/16

(52) U.S. Cl. 435/5; 424/188.1; 530/350

(57) ABSTRACT

Materials and methods of use thereof are presented for the treatment or diagnosis of HIV. The materials are aptamers that bind to gp41, CCR5 or other HIV viral targets that are involved in the formation of the gp41 fusion-active intermediate.

### REFERENCE TO RELATED APPLICATIONS

This application is related to and claims priority to U.S. Provisional Patent Application Ser. No. 60/415,390 filed Oct. 2, 2002; U.S. Provisional Patent Application Ser. No. 60/441,416 filed Jan. 21, 2003; U.S. Provisional Patent Application Ser. No. 60/461,966 filed Apr. 10, 2003; and U.S. Provisional Patent Application Ser. No. 60/465,148 filed Apr. 23, 2003, each of which is herein incorporated by reference in its entirety.

### FIELD OF THE INVENTION

The invention relates generally to the field of nucleic acids and more particularly to compositions and methods for diagnosing, treating or preventing HIV with aptamers or aptamer compositions that specifically bind to gp41.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (MAbs), are capable of specifically binding to selected targets and, through binding, block their targets' ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc.) that drive affinity and specificity in antibody-antigen complexes.

Aptamers have a number of desirable characteristics for use as therapeutics including high specificity and affinity, biological efficacy, and excellent pharmacokinetic properties. In addition, they offer specific competitive advantages over antibodies and other protein biologics, for example:

1) Speed and Control.

Aptamers are produced by an entirely in vitro process, allowing for the rapid generation of initial (therapeutic) leads. In vitro selection allows the specificity and affinity of the aptamer to be tightly controlled and allows the generation of leads against both toxic and non-immunogenic targets.

2) Toxicity and Immunogenicity.

Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).

3) Administration.

Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/ml) and comparatively low molecular weight (aptamer: 10-50 KD; antibody: 150 KD), a weekly dose of aptamer may be delivered by injection in a volume of less than 0.5 ml. Aptamer bioavailability via subcutaneous administration is >80% in monkey studies (Tucker, 1999). In addition, the small size of aptamers allows them to penetrate into areas of conformational constrictions that do not allow for antibodies or antibody fragments to penetrate, presenting yet another advantage of aptamer-based therapeutics or prophylaxis.

4) Scalability and Cost.

Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale synthesizer can produce upwards of 100 kg oligonucleotide per year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability.

Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

The human immunodeficiency virus (HIV), the cause of acquired immunodeficiency syndrome (AIDS), remains an extremely serious threat to public health worldwide. Globally, over 40 million people are infected with HIV, with roughly 14,000 new infections arising each day (Report to UN on AIDS, 2002). Along with efforts to find an as yet elusive prophylactic treatment, the best long-term solution for controlling morbidity of the current AIDS epidemic is development of a safer and more effective HIV therapeutic for treatment of infected individuals.

Among approaches for searching for therapeutics are efforts to inhibit the fusion of viral particles to cell membranes through viral glycoprotein-cell membrane-bound receptor interactions. The mature HIV envelope glycoprotein exists as a trimer that arises through processing of a larger precursor (gp160) to gp120 and gp41 components which non-covalently associate on the virion surface (Kowalski, et al., 1987; Lu et al., 1995; Burton, 1997). The gp120 subunit is the primary viral antigen against which humoral immune responses are mounted (Profy, 1990; reviewed in Poignard et al., 2001). The gp120 envelope protein is responsible for binding to the CD4 cell-surface receptor and a chemokine co-receptor, CCR5 or CXCR4 (Broder et al., 1996; D'Souza et al., 1996; Wilkinson, 1996). See FIG. 2.

Following gp120-mediated receptor binding, the viral membrane-anchored gp41mediates fusion of the viral and target cell membranes. The gp41 ectodomain contains a hydrophobic glycine-rich fusion peptide (amino acids 512-527, numbering based on XHB2 gp160 variant as described in Chan et al., 1997) at the amino terminus, which is essential for membrane fusion. Following the fusion peptide are two helical hydrophobic repeats defined by a heptad repeat (abcdefg)ₙ, where the residues occupying the a and d positions are predominantly hydrophobic. These two heptad repeats, referred to as the N36 (residues 546-581) and C34 (residues 628-661) peptides, are shown in FIG. 3 (figure adapted from Chan et al. 1997). A loop region containing a disulfide linkage separates the two heptad repeat regions. Following the second heptad repeat is a six residue sequence (amino acids 662-667) recognized by monoclonal antibody 2F5 (Muster et al., 1993; Conley et al., 1994). The region of the gp41ectodomain proximal to the viral membrane is abundant in the amino acid tryptophan and has been shown to be critical for the membrane fusion mechanism of HIV-1 (Salzwedel et al., 1999; Suarez et al., 2000; Schibli et al., 2001).

HIV gp41 exists in two distinct conformations, a native or nonfusogenic state or conformation and a fusion active state or conformation (Chan et al., 1998). On the surface of free virions, gp41 exists in the native state or conformation with the N-terminal fusion peptide inaccessible. Following interaction of the gp120/gp41 complex with cell-surface receptors, gp41 undergoes a series of conformational changes leading to the formation of the fusion-active conformation and, subsequently, fusion of the viral and target cell membranes (Chan et al., 1998). More specifically, these conformational changes involve the exposure of the fusion peptide and its insertion into the target membrane (to form the fusion intermediate) followed by the formation of a hairpin-like structure (the fusion active conformation) which brings the viral and target membranes into proximity allowing viral entry into the target cell (Chan et al., 1998; McGaughey et al., 2003). Crystallographic analysis has demonstrated that the gp41 fusion-active core adopts a six-stranded helical bundle (Chan et al., 1997). Three N-terminal peptides adopt a homo-trimeric helical coiled-coil structure forming the center of the bundle. Three C-terminal peptide helices pack into hydrophobic grooves on the outer surface of the N-peptide core in an antiparallel manner forming a trimer-of-hairpins structure. The trimer-of-hairpins structure likely resembles the fusion-active conformation since this structural motif brings the N-terminal region of gp41 containing the fusion peptide together with the C-terminal region that is anchored to the viral membrane (Chan et al. 1998; Root et al, 2001). This conformational change brings the viral and target cell membranes together, promoting fusion.

Peptide molecules that interact with either the N-terminal or C-terminal heptad repeat domains have been shown to inhibit viral fusion (Wild et al., 1994; Judice et al., 1997; Jiang et al., 1993, Eckert et al. 1999). These peptides are thought to inhibit infection by binding to gp41 and preventing the conformational changes that result in the formation of the hairpin-like structure required for viral fusion. One peptide that has been tested in clinical trials, DP178 (trade name FUZEON™), as shown in FIG. 3, is derived from the C-terminal region of the gp41 (residues 638-673) and successfully blocks viral membrane fusion in vitro (Wild et al., 1994; Lawless et al., 1996; Kilby et al., 1998). However, peptides inherently lack many of the desirable qualities of useful pharmaceutical therapeutics such as stability and oral bioavailability.

Thus, there is a long-felt and yet unmet need for a therapeutic agent that can bind specifically to gp41 and inhibit conformational changes in gp41 that result in the fusion of the viral and cell membranes to prevent or treat infection of cells by HIV. Aptamers specific for gp41 meet these and other needs.

### BRIEF DESCRIPTION OF THE DRAWINGS

FIG. 1 shows the in vitro aptamer selection (SELEX™) process from pools of random sequence oligonucleotides.

FIG. 2 shows a schematic of HIV infection of cells upon CD4 induced binding of gp120 to CCR5 membrane protein.

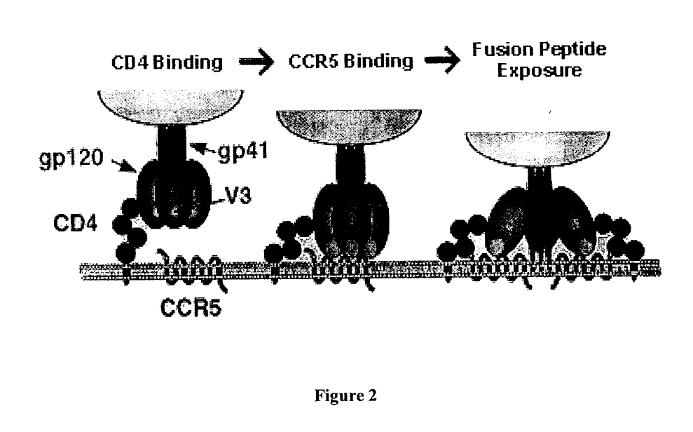

FIG. 3A shows a schematic of HIV gp41 functional regions including the fusion peptide (FP), the two heptad repeats N36 and C34, the region spanning the 2F5 epitope (residues 661-684), the transmembrane region (TM), and the cytoplasmic domain (CYTO). FIG. 3B shows the N36 heptad repeat 1 sequence detail showing residues L and W critical for membrane fusion activity in bold and underlined, and C34 heptad repeat 2 sequence detail showing W, W and I residues that make hydrophobic contacts in pocket in bold and underlined.

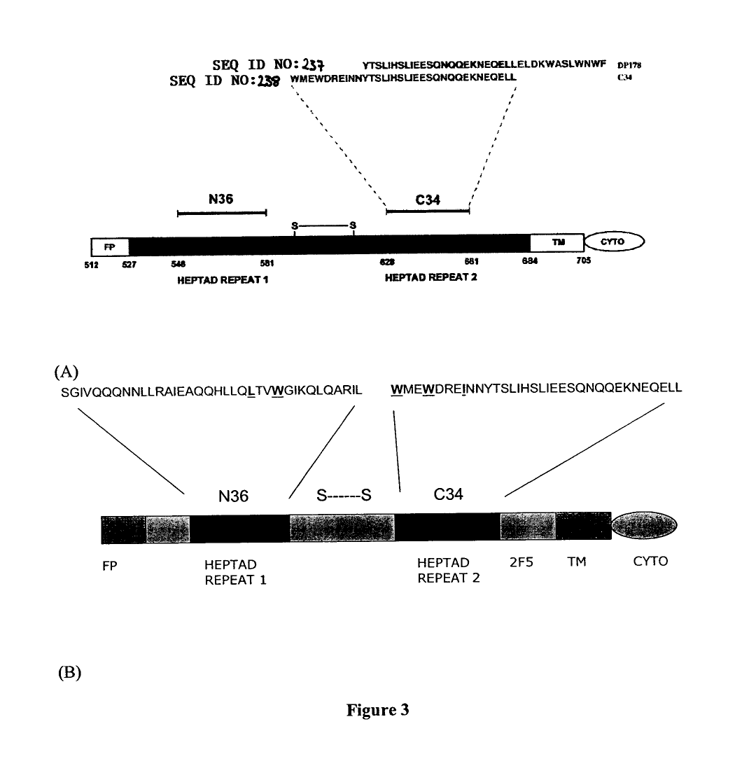

FIG. 4 shows a schematic of the steps typically required to generate an aptamer.

FIG. 5 shows a schematic of a working model of the process of HIV entry into cells.

FIGS. 6 (A) and (B) shows histograms of gp41 candidate binding to three targets.

### SUMMARY OF THE INVENTION

In one embodiment, the present invention provides aptamers or aptamer compositions which bind to gp41.

In a preferred embodiment, the present invention provides aptamers or aptamer compositions which bind to the N36 or C34 regions of gp41.

In one embodiment, the present invention provides a method of treating or preventing HIV infection using aptamers or aptamer compositions. In a preferred embodiment, the aptamers or aptamer compositions bind to the N36 or C34 regions of gp41 preventing gp41 from undergoing a conformational shift which brings the N36 regions and C34 regions together to fuse the viral and cell membranes.

In one embodiment, the present invention provides a method of diagnosing HIV infection using aptamers or aptamer compositions. In a preferred embodiment, the aptamers or aptamer compositions bind to the N36 or C34 regions of gp41.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

The SELEX™ Method

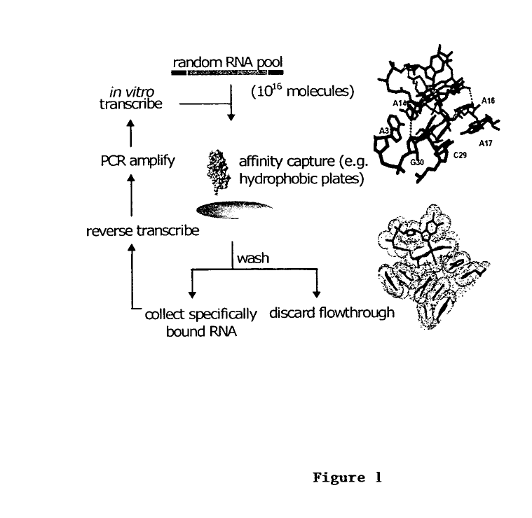

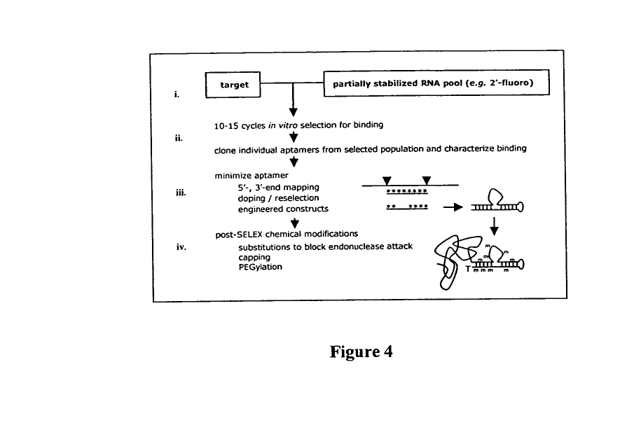

A suitable method for generating an aptamer to gp41 is with the process entitled “Systematic Evolution of Ligands by EXponential Enrichment” (“SELEX™”) generally depicted in FIGS. 1 and 4. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotide templates comprising randomized sequences derived from chemical synthesis on a standard DNA synthesizer. In some examples, a population of 100% random oligonucleotides is screened. In others, each oligonucleotide in the population comprises a random sequence and at least one fixed sequence at its 5′ and/or 3′ end which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores (described further below), sites for selective binding to affinity columns, and other sequences to facilitate cloning and/or sequencing of an oligonucleotide of interest.

The random sequence portion of the oligonucleotide can be of any length and can comprise ribonucleotides and/or deoxyribonucleotides and can include modified or non-natural nucleotides or nucleotide analogs as described, e.g., in U.S. Pat. Nos. 5,958,691; 5,660,985; 5,958,691; 5,698,687; 5,817,635; and 5,672,695, PCT publication WO 92/07065. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

Template molecules typically contain fixed 5′ and 3′ terminal sequences which flank an internal region of 30-50 random nucleotides. A standard (1 μmole) scale synthesis will yield 10¹⁵-10¹⁶ individual template molecules, sufficient for most SELEX experiments. The RNA library is generated from this starting library by in vitro transcription using recombinant T7 RNA polymerase. This library is then mixed with the target under conditions favorable for binding and subjected to step-wise iterations of binding, partitioning and amplification, using the same general selection scheme, to achieve virtually any desired criterion of binding affinity and selectivity. More specifically, starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

Within a nucleic acid mixture containing a large number of possible sequences and structures, there is a wide range of binding affinities for a given target. A nucleic acid mixture comprising, for example a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX procedures with contiguous randomized segments be initiated with nucleic acid sequences containing a randomized segment of between about 20-50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. For example, U.S. Pat. No. 5,580,737 discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

One potential problem encountered in the use of nucleic acids as therapeutics and vaccines is that oligonucleotides in the phosphodiester form may be quickly degraded in body fluids by intracellular and extracellular enzymes such as endonucleases and exonucleases before the desired effect is manifest. The SELEX method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described, e.g., in U.S. Pat. No. 5,660,985, which describes oligonucleotides containing nucleotide derivatives chemically modified at the 5′ and 2′ positions of pyrimidines. U.S. Pat. No. 5,756,703 describes oligonucleotides containing various 2′-modified pyrimidines. U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping. In preferred embodiments of the instant invention, the nucleic acid ligands are RNA molecules that are 2′-O-methyl (2′-OMe) or 2′-fluoro (2′-F) modified on the sugar moiety of pyrimidine residues.

The modifications can be pre- or post-SELEX process modifications. Pre-SELEX process modifications yield nucleic acid ligands with both specificity for their SELEX target and improved in vivo stability. Post-SELEX process modifications made to 2′-OH nucleic acid ligands can result in improved in vivo stability without adversely affecting the binding capacity of the nucleic acid ligand.

Other modifications are known to one of ordinary skill in the art. Such modifications may be made post-SELEX process (modification of previously identified unmodified ligands) or by incorporation into the SELEX process.

The SELEX method encompasses combining selected oligonucleotides with other selected oligonucleotides and non-oligonucleotide functional units as described in U.S. Pat. No. 5,637,459 and U.S. Pat. No. 5,683,867. The SELEX method further encompasses combining selected nucleic acid ligands with lipophilic or non-immunogenic high molecular weight compounds in a diagnostic or therapeutic complex, as described in U.S. Pat. No. 6,011,020. VEGF nucleic acid ligands that are associated with a lipophilic compound, such as diacyl glycerol or dialkyl glycerol, in a diagnostic or therapeutic complex are described in U.S. Pat. No. 5,859,228.

VEGF nucleic acid ligands that are associated with a lipophilic compound, such as a glycerol lipid, or a non-immunogenic high molecular weight compound, such as polyalkylene glycol are further described in U.S. Pat. No. 6,051,698. VEGF nucleic acid ligands that are associated with a non-immunogenic, high molecular weight compound or a lipophilic compound are further described in PCT Publication No. WO 98/18480. These patents and applications allow the combination of a broad array of shapes and other properties, and the efficient amplification and replication properties, of oligonucleotides with the desirable properties of other molecules.

The identification of nucleic acid ligands to small, flexible peptides via the SELEX method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). The use of 2-fluoro-ribonucleotide oligomer molecules can increase the sensitivity of a nucleic acid molecule for a target molecule by ten-to-one hundred-fold over those generated using unsubstituted ribo- or deoxyribo-oligonucleotides (Pagratis, et al., Nat. Biotechnol. 15:68-73 (1997)), providing additional binding interactions with a target molecule and increasing the stability of the secondary structure(s) of the nucleic acid molecule (Kraus, et al., Journal of Immunology 160:5209-5212 (1998); Pieken, et al., Science 253:314-317 (1991); Lin, et al., Nucl. Acids Res. 22:5529-5234 (1994); Jellinek, et al. Biochemistry 34:11363-11372 (1995); Pagratis, et al., Nat. Biotechnol 15:68-73 (1997)).

Nucleic acid aptamer molecules are generally selected in a 5 to 20 cycle procedure. In one embodiment, heterogeneity is introduced only in the initial selection stages and does not occur throughout the replicating process.

gp41 Aptamers

The current invention describes aptamers that bind to gp41. In one embodiment, the gp41 aptamers or aptamer compositions can be used alone or in conjunction with other anti-retroviral therapeutics as a therapeutic “cocktail” to treat HIV infection in subjects. The gp41 aptamers disclosed herein can be chemically synthesized or transcribed from DNA templates using standard techniques for oligonucleotide synthesis and/or PCR.

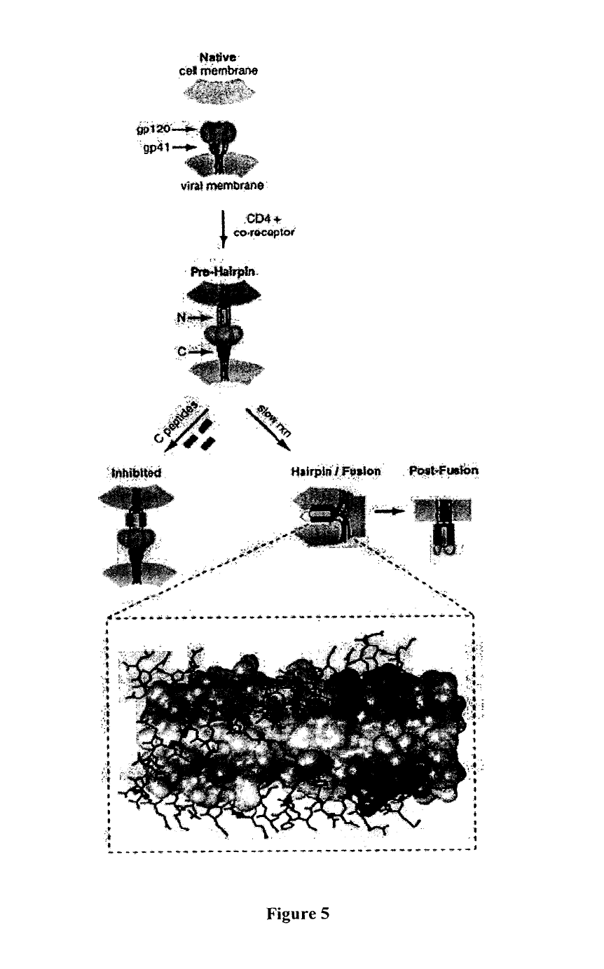

Infection of cells by HIV-1 requires fusion of the cellular and viral membranes, a process mediated by the viral envelope glycoprotein complex (gp120/gp41) and cell surface receptors on the target cell. FIG. 5 shows a schematic of a working model of the HIV entry process (Chan et al., 1998). Binding of gp120/gp41 complex to cellular receptors (CD4 and a chemokine co-receptor such as CCR-5 or CXCR-4) induces a conformational change in the envelope glycoprotein. A transient species results, called the prehairpin (or fusion) intermediate, in which gp41 exists as a membrane protein simultaneously in both viral and cellular membranes (Chan et al., 1998). The prehairpin intermediate resolves to a trimer-of-hairpins structure that likely represents the fusion-active state of gp-41 (Blacklow et al, 1995) as seen in the X-ray crystal structure of a protease-resistant core of gp41 (Chan et al, 1997). The trimer-of-hairpins structure is a common feature of diverse viral membrane fusion proteins (Singh et al., 1999). It is unclear whether hairpin formation occurs before, or simultaneously with, the actual fusion of the two bilayers (Eckert et al, 1999).

In gp41, a central three-stranded coiled coil (formed by the N-terminal regions of gp41) is surrounded by helices derived from the C-terminal end of gp41 ectodomains, packed in an antiparallel manner around the outside of the coiled coil (FIG. 5, inset). Peptides corresponding to these regions of gp41 are referred to as N-peptides and C-peptides, respectively. The N peptide coiled coil trimers are formed by three central N-peptides and three helical C-peptides pack along conserved grooves on the surface of the coiled-coil trimer. There are three symmetry-related hydrophobic pockets on the surface of the N-peptide coiled coil. This pocket region is highly conserved among HIV-1 isolates. The lining of the hydrophobic pocket is comprised of 11 amino acid residues (Chan et al., 1997). These conserved 11 residues are: Leu-565, Leu-566, Leu-568, Thr-569, Val-570, Trp-571, Gly-572, Ile-573, Lys-574, Leu-576, and Gln-577 of HSB2 (Chan et al., 1997, Eckert et al., 1999).

Aptamers useful as HIV therapeutics can be isolated on the basis of their ability to inhibit the ability of gp41 to promote fusion of the viral and infected cell membranes. To do this, aptamers can be generated against gp41 itself (preferably, against regions of the N36 or C34 peptides and even more preferably against regions of the N36 or C34 peptides containing residues believed to be critical for membrane fusion or the 2F5 epitope) or to other viral targets (e.g., CCR5 or gp120) involved in the transformation from the fusion inactive to the fusion active intermediate and/or involved in the conformational change undertaken by the fusion active intermediate to bring the viral and infected cell membranes together. With regard to gp41, the SELEX process can be performed using gp41, gp41 peptides, or gp41 peptide inhibitors as targets to select aptamers that bind to gp41 and inhibit the biological activity of gp41. It is believed that gp41 aptamers can inhibit the biological activity of gp41 by, e.g., binding to the N36 and/or C34 regions of the fusion active intermediate and preventing gp41 from undergoing the conformational change necessary to bring about membrane fusion.

HIV specific aptamers or aptamer compositions, including gp41 aptamers or aptamer compositions, can also be used to deliver a toxic payload to the vicinity of the virus. In still other applications, HIV specific aptamers or aptamer compositions, including gp41 aptamers or aptamer compositions, can be used as diagnostics.

gp41 Aptamer-Toxin Conjugates

One use of the gp41 specific aptamers of the present invention is in the selective delivery or targeting of toxic agents linked to the aptamers. The linked cytotoxic agent is thus brought in close proximity of the HIV or HIV infected cells, which allows for the cytotoxic agent to exert its cytotoxic effects on the virus and/or infected cell.

In one aspect, the toxin is a chemotoxin. In some embodiments, the toxin is a protein toxin. In other embodiments, the toxin is a nucleic acid toxin.

In some embodiments, the toxin is attached to the aptamer through covalent bond. If desired, the toxin is attached to an aptamer through a hydrolysable bond, and/or through a bond that can be cleaved through enzymatic activity. In other embodiments, the toxin is attached to the aptamer through a non-covalent bond.

In some embodiments, the aptamer-toxin conjugate binds to target, thereby delivering toxin to the vicinity of the target. The toxin may interact with the same target, or with a second target in the vicinity of the first target.

In some embodiments, the aptamer-toxin conjugate binds to a target, and binding to target results in a change in conformation of the aptamer-toxin. The change in conformation results in a change in activity of the aptamer-toxin.

For example, in some embodiments, binding of the aptamer-toxin conjugate to a target can result in a change in conformation of the aptamer-toxin conjugate, such change resulting in a release of the toxin.

Alternatively, or in addition, binding of the aptamer-toxin conjugate to a target can result in a change in conformation of the aptamer-toxin conjugate, wherein the conformational change results in an activation of the toxin.

In a further embodiment, the aptamer-toxin conjugate binds to a target, where binding to target results in a change in conformation of the aptamer-toxin conjugate, and the change results in inactivation of the toxin.

In various embodiments, an aptamer-toxin conjugate is provided whose half-life is less than, equal to, or greater than, the half-life of the toxin.

Also provided by the invention is a method of generating an aptamer-toxin conjugate that includes attaching a toxin to an aptamer. In some embodiments, the aptamer in the moiety is created using a process termed “Systematic Evolution of Ligands by EXponential Enrichment” (the “SELEX process”). The SELEX process is a method for the in vitro evolution of nucleic acid molecules with highly specific binding to target molecules and is described in, e.g., U.S. Pat. No. 5,475,096 entitled “Nucleic Acid Ligands”, and U.S. Pat. No. 5,270,163 (see also WO91/19813) entitled “Nucleic Acid Ligands”.

For example, the invention includes a method of generating an aptamer-toxin conjugate by attaching a toxin to a random pool of nucleic acids and then using the SELEX process to find the optimized aptamer-toxin conjugate from within the random pool. Alternatively, can attach toxin to aptamer post-selection.

In some embodiments, the method of generating an aptamer-toxin conjugate results in a aptamer whose half-life is engineered to match the half life of the toxin. For example, the invention includes a method of generating an aptamer-toxin conjugate where the aptamer half life is engineered to match the half life of the toxin by adjusting the percentage of nuclease resistant bases in the aptamer. In other embodiments, the invention includes a method of generating an aptamer-toxin conjugate where the aptamer half life is engineered to match the half life of the toxin by changing the 5′ and/or 3′ end capping.

Toxins useful in the present invention include chemotoxins having cytotoxic effects. These can be classified in their mode of action: 1) tubulin stabilizers/destabilizers; 2) anti-metabolites; 3) purine synthesis inhibitors; 4) nucleoside analogs; and 5) DNA alkylating or modifying agents. Radioisotopes also have cytotoxic effects useful in the present invention.

Examples of suitable toxins include, e.g., chemotherapeutic agents. Chemotherapeutics are typically small chemical entities produced by chemical synthesis and include cytotoxic drugs, cytostatic drugs as well as compounds which affects cells in other ways such as reversal of the transformed state to a differentiated state or those which inhibit cell replication. Examples of chemotherapeutics include, but are not limited to: methotrexate (amethopterin), doxorubicin (adrimycin), daunorubicin, cytosinarabinoside, etoposide, 5-4 fluorouracil, melphalan, chlorambucil, and other nitrogen mustards (e.g., cyclophosphamide), cis-platinum, vindesine (and other vinca alkaloids), mitomycin and bleomycin.

Toxins can include complex toxic products of various organisms including bacteria, plants, etc. Examples of toxins include but are not limited to: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin. Protein toxins may be produced using recombinant DNA techniques as fusion proteins which include peptides of the invention. Protein toxins may also be conjugated to compounds of the invention by non-peptidyl bonds. In addition, photosensitizers and cytokines can also be used with the present invention.

Cytotoxic molecules that can be used in the present invention are anthracycline family of cytotoxic agents, e.g., doxorubicin (DOX). Doxorubicin damages DNA by intercalation of anthracycline protion, metal ion, chelation, or by generation of free radicals. DOX has also been shown to inhibit DNA topoisomerase II. Doxorubicin has been show clinically to have broad spectrum of activity and toxic side effects that are both dose-related and predictable. Efficacy of DOX is limited by myelosuppression and cardiotoxicity. Complexed with a targeting moiety such as an aptamer increases intratumoral accumulation while reducing systemic exposure.

Maytansinoids are very toxic chemotherapeutic molecules that can be used as therapeutic moieties of the present invention. Maytansinoids effect their cytotoxicity by inhibiting tubulin polymerization, thus inhibiting cell division and proliferation. Maytansinoid derivative DM1 has been conjugated to other targeting moieties, e.g., murine IgG1 mAb against MUC-1 and to an internalizing anti-PSMA murine monoclonal antibody 8D11 (mAb) through disulfide linker chemistry.

Enediynes are another class of cytotoxic molecules that can be used as therapeutic moieties of the present invention. Enediynes effect their cytotoxicity by producing double-stranded DNA breaks at very low drug concentrations. The enediynes class of compounds includes calicheamicins, neocarzinostatin, esperamicins, dynemicins, kedarcidin, and maduropeptin. Linking chemistries for these compounds include periodate oxidation of carbohydrate residues followed by reaction with a hydrazide derivative of calicheamycin, for example. These conjugates utilize an acid-labile hydrazone bond to a targeting moiety, such as a monoclonal antibody to ensure hydrolysis following internalization into lysosomes, and a sterically protected disulfide bond to calicheamicin to increase stability in circulation.

Tumor therapeutics also include radionuclides, particularly high energy alpha particle emitters. Alpha particles are high energy, high linear energy transfer (LET) helium nuclei capable of strong, yet selective cytotoxicity. Approximately 100 radionuclides decay with alpha emission. A single atom emitting an alpha particle can have a lethal cytotoxic effect on a single cell. Conjugates of radionuclides to mAbs have been used in preclinical models of leukemia and prostate cancer, and a phase I clinical trial is underway with ²¹¹At-labeled anti tenascin mAb against malignant gliomas.

Radioisotopes may be conjugated to compounds of the invention. Examples of radioisotopes which are useful in radiation therapy include, e.g., ⁴⁷Sc, ⁶⁷Cu, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi. Some alpha particle emitting radioisotopes exhibit too short a half life to be effective therapeutics against most tumors. For example, ²¹³Bi has a 46 minute half life which limits its efficacy to only the most accessible cancer cells, and poses practical obstacles such as timely shipment and administration. Another radioisotope ²²⁵Ac is a more suitable radiotherapeutic because each ²²⁵Ac atom decays into several daughter atoms, four of which also emits alpha particles.

The gp41 specific aptamers of the present invention can be attached to therapeutic moieties , e.g., toxins, using methods known in the art. For example, methods for generating blended nucleic acid ligands comprised of functional unit(s) added to provide a nucleic acid ligand with additional functions are described in U.S. Pat. No. 5,683,867, U.S. Pat. No. 6,083,696, and U.S. Pat. No. 5,705,337. The latter patent discloses methods for identifying nucleic acid ligands capable of covalently interacting with targets of interest. The nucleic acids can be associated with various functional units. The method also allows for the identification of nucleic acids that have facilitating activities as measured by their ability to facilitate formation of a covalent bond between the nucleic acid, including its associated functional unit, and its target.

Cytotoxics—Small Organic Molecule Linking Chemistries

To link nucleic acid aptamers of the present invention to small molecule cytotoxic agents that contain carboxylate groups, the latter are converted into an amine-reactive probe (e.g. NHS ester) by conventional synthetic organic reactions, and then coupled to an amine oligonucleotide aptamer. Amine-containing small molecules can be coupled to an activated oligo (e.g. 5′-carboxy-modifier C10 (Glen Research) according to the Glen technical product bulletin). Alternatively, an amine-oligo can be activated in situ by crosslinking reagents, including but not limited to DSS, BS³ or related reagents (Pierce, Rockford, Ill.), and further coupled to amines.

Thiol-containing small molecules can be coupled to 2,2-dithio-bispyridine activated thiol aptamer or an SPDP-activated (Pierce, Rockford, Ill.) amine-oligo.

Small molecules that do not contain carboxylate, amine or thiol groups are preferably converted into such by conventional synthetic organic chemistry by methods known to those of skill in the art.

Additionally, encapsulated (e.g. in liposomes) cytotoxics can also be linked to aptamers or riboreporters of the present invention with acid-labile linkers, enzyme clavable linkers used in the art for linking liposome to reactive moieties, such as activated oligonucleotides.

Acid-labile linkers include for illustration but not limitation, cis-aconityl linkers used to link anthracyclines, doxorubicin (DOX) or daunorubicin (DNR), to immunoconjugates such as several mAbs (e.g., anti-melanoma mAb 9.927); leading to released cytotoxic agents in the environment of lysozomes.

Hydrazone linkers have been used to conjugate small molecule cytotoxic agents including DNR, morpholino-DOX to anti-αvβ3 mAb LM609, and anti-Le^{y} mAb BR96. These hydrazone linkers are acid labile at pH 4.5. Other acid-sensitive anthracycline conjugates have been obtained through modification of the C-13 carbonyl group to give acylhydrazone, semicarbazones, thiosemicarbazones and oximes.

Cytotoxics—Peptides (Synthetic) Linking Chemistries

In the case of peptide cytotoxic agents, methods for coupling of synthetic peptides include synthesis of an amine-reactive activated ester (e.g., NHS) of the peptide, coupling to amine-oligo.

Another method of linking peptide cytotoxic moieties to the targeting moieties of the present invention also include synthesis of a cytotoxic peptide moiety with an extra C- or N-terminal cysteine. This can be activated with 2,2-dithio-bispyridine and coupled to a thiol-modified aptamer oligo (standard automated synthesis, final coupling with an thiol-modifier [Glen Research, Sterling, Va.]). Alternatively, the thiol-modified aptamer is activated with 2,2-dithio-bispyridine and coupled to the cys-peptide. Lastly, an amino-terminated oligo can be activated with SPDP (Pierce, Rockford, Ill.) and coupled to the cys-containing peptide. All three methods generate the conjugate coupled through a disulfide bond.

Another method of linking peptide cytotoxic moieties to the targeting moieties of the present invention also includes modification of a targeting moiety consisting of an amine-oligo with a maleimide reagent, e.g., GMBS, (Pierce, Rockford, Ill.), subsequent coupling to cys-peptide.

Another method of linking peptide cytotoxic moieties to the targeting moieties of the present invention also includes synthesis of a targeting moiety consisting of an oligo modified with 5′-carboxy-modifier C10 (Glen Research) and in-situ coupling to an amine-containing molecule (i.e. peptide) according to methods known in the art.

Another method of linking peptide cytotoxic moieties to the targeting moieties of the present invention also includes oxidizing 3′-ribo-terminated oligos with sodium meta-periodate and the resulting aldehyde reacted with amine peptides in the presence of reducing agents. In addition, C-terminal peptide hydrazides can couple to an oxidized RNA even without the aid of reducing agents.

Cytotoxics—Protein Linking Chemistries

Methods of linking cytotoxic protein moieties of the present invention to targeting moieties of the present invention are principally the same as those methods used for linking peptides.

Methods of linking protein cytotoxic protein moieties of the present invention include activation of the targeting moiety of the invention consisting of an amino-terminated oligo with e.g. SPDP or GMBS (Pierce, Rockford, Ill.), or of an thiol-oligo with 2,2-dithio-bispyridine and coupling to the cys-containing protein.

Another method of linking cytotoxic protein moieties of the invention with targeting moieties of the present invention include coupling of protein amines to an amine-containing oligo using crosslinking reagents, e.g., DSS, BS³ or related reagents (Pierce, Rockford, Ill.).

Radioisotopes Cytotoxic Moieties Linking Chemistries

Methods of linking cytotoxic moieties of the present invention consisting of radioactive metal ions (e.g., isotopes of Tc, Y, Bi, Ac, Cu etc.) to targeting moieties of the present invention include chelation with a suitable ligand, such as DOTA (Lewis, et al., Bioconjugate Chemistry 2002, 13, 1178). A generic labeling scheme would start with the synthesis of a 5′-amino-modified aptamer oligo (standard automated synthesis, final coupling with an amino-modifier [Glen Research, Sterling, Va.]). Then, the chelator is converted into an amine-reactive activated ester, and subsequently coupled to the oligo similar to the method described in Lewis, et al.

Another method of linking radionuclide cytotoxic moieties of the present invention to targeting moieties of the present invention include oxidizing 3′-ribo-terminated oligos with sodium meta-periodate and the resulting aldehyde reacted with amine-containing chelators or radiolabels in the presence of reducing agents. Alternatively, hydrazine, hydrazide, semicarbazide and thiosemicarbazide derivatives of chelators or radiolabels can be used.

Administration, Dose and Treatment Regimes

The method for preventing HIV infection or reducing the levels of HIV in infected individuals involves administering to a human an aptamer or aptamer composition that inhibits conformational changes in gp41 preventing/impairing the ability of HIV to infect cells in vivo. For already infected individuals, the method will decrease the levels of circulating virus (“viral load”), ameliorating the effects of the disease.

The terms “treating,” “treatment,” and the like are used herein to mean obtaining a desired pharmacologic or physiologic effect. The effect can be prophylactic in terms of completely or partially preventing a disorder or sign or symptom thereof, or can be therapeutic in terms of a partial or complete cure for a disorder and/or adverse effect attributable to the disorder. “Treating” as used herein covers any treatment and includes: (a) preventing a disorder from occurring in a subject that can be predisposed to a disorder, but has not yet been diagnosed as having it; (b) inhibiting the disorder, i.e., arresting its development; or (c) relieving or ameliorating the disorder. An “effective amount” or “therapeutically effective amount” is the amount sufficient to obtain the desired physiological effect. Appropriate dosing regimens for the vaccine is generally determined on the basis of controlled clinical trials across patient populations; the effective amount for the vaccine is selected by the physician in each case on the basis of factors normally considered by one skilled in the art to determine appropriate dosages, including the age, sex, and weight of the subject to be treated, the condition being treated, and the severity of the medical condition being treated.

Administration of gp41Aptamer Therapeutic

The gp41 aptamer therapeutic may be formulated and administered through a variety of means, including systemic, localized or topical administration. Preferably, the gp41 aptamer therapeutic is formulated and administered systemically. Techniques for formulation and administration may be found in “Remington: The Science and Practice of Pharmacy, Twentieth Edition,” Lippincott Williams & Wilkins, Philadelphia, Pa. Suitable routes may include but are not limited to oral, rectal, transmucosal or intestinal administration; parenteral delivery, including intramuscular or subcutaneous injections; or intranasal injections.

For systemic administration, injection is preferred, including intramuscular (preferred) and subcutaneous. For injection, the therapeutics are formulated in aqueous solutions, preferably in physiologically compatible buffers such as Hanks's solution, Ringer's solution, or physiological saline buffer and may include adjuvants (e.g., alums, polymers, copolymers). In addition, the therapeutic may be formulated in solid or lyophilized form, then redissolved or suspended immediately prior to use. Dose, dosing interval and number of doses will depend upon the patient population (varying by age, weight, underlying diseases, immunologic status etc.).

The aptamer therapeutics may be administered to patients alone or in combination with other therapies. Such therapies include the sequential or concurrent administration of small molecule anti HIV protease and reverse transcriptase inhibitors or antagonists and/or other anti-HIV vaccines that work through different mechanisms (e.g., by generating T-cell-mediated immunity).

Pharmaceutical Compositions

Pharmaceutical compositions suitable for administration will typically comprise the therapeutic aptamer and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in “Remington: The Science and Practice of Pharmacy, Twentieth Edition,” Lippincott Williams & Wilkins, Philadelphia, Pa. Preferred examples of such carriers or diluents include, but are not limited to, water, saline, Ringer's solutions, dextrose solution and phosphate buffered solutions. Adjuvants such as aluminum phosphate, liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intramuscular and subcutaneous, administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. Immunogenicity may be enhanced by the inclusion of adjuvants such as alum or other agents commonly known in the field. The parenteral preparation can be enclosed in ampoules, disposable syringes or multiple dose vials made of glass or plastic. In all cases, the composition must be sterile and should be fluid to the extent that easy syringeability exists. It must be stable under the conditions of manufacture and storage and if formulated in multi-dose vials must be preserved against the contaminating action of microorganisms such as bacteria and fungi.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying, lyophilization and freeze-drying that yields a powder of the active ingredient plus any additional desired ingredient from a previously sterile-filtered solution thereof.

It is especially advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### Pool Preparation for gp41 Aptamer Selection Against N36 Peptide Targets

Selections for gp41 specific binding aptamers were performed to generate aptamers against HIV-1 gp41 peptides using a variety of different pools containing 2′-fluoro-modified UTP and CTP nucleotides. Two different pools were originally used in selection against the gp41 N terminal (N-17) peptide: the Yang N40 pool (jd6054a), template and primers shown in Table 1, and the semi-structured N40 pool (jd6093e), template and primers shown in Table 2. In addition to the N-17 gp41peptide, a GCN4 peptide and a single mutant N-terminal (N17) gp41 peptide were used, as shown in Table 6.

In the doped re-selection, three pools were used to generate tighter binding aptamers: the type I doped pool (jd10386a), the type II doped pool (jd10386b), and the type III doped pool (jd10381a). Pool templates include two oligonucleotides of defined sequence separated by a randomized region of nucleotides, e.g., 30 or 40 nucleotides in length (N30 or N40).

TABLE 1
Yang N40 pool (Jd6054a).

a. Pool template (jd6054a).
SEQ ID NOs. 1 & 2 jd6054a:

(SEQ ID NO:1)

5′-GGAGACAAGAAUAAACGCUCAA-3′

(SEQ ID NO:2)

-(N40)-5′-UUCGACAGGAGGCUCACAACAGGC-3′
b. Pool primers (jd6011a/jd6011b),
SEQ ID NO.3 jd6011a-5′primer:
5′-TAATACGACTCACTATAGGGAGACAAGAATAAACGCTCAA-3′
SEQ ID NO.4 jd6011b-3′primer:
5′-GCCTGTTGTGAGCCTCCTGTCGAA-3′

The Yang N40 pool and primers described in Table 1 were synthesized using an ABI Expedite 8909 synthesizer and deprotected using standard methods. The pool was then PolyPac purified. The pool was quantified, and large-scale PCR was performed to achieve a 3×10¹⁵ RNA molecule pool diversity. The PCR product was then in vitro transcribed overnight using 2′-fluoro modified UTP and CTP nucleotides (Durascribe Kit). Following transcription, the RNA pool was DNase treated, EtOH precipitated, and then gel purified. The gel slices were then electro-eluted and EtOH precipitated. The final pool concentration was 83.4 uM (2.51×10¹⁶ total RNA molecules), containing enough for eight selection pools. The pool was then tested for its ability to be reverse-transcribed and PCR amplified.

TABLE 2
Semi-structured N40 pool (jd6093e).

a. Pool template (jd6093e).

| | |
|---|---|
| SEQ ID NOs.5 & 6 jd6093e, | |
| 5′-GGAGCCTTCCTCCGGA-3′ | (SEQ ID NO:5) |
| (N40) 5′-TCCGGTTTCCCGAGCTT-3′ | (SEQ ID NO:6) |

b. Pool primers (jd6093a/jd6093b).
SEQ ID NO.7 jd6093a-5′primer:
5′-TAATACGACTCACTATAGGAGCCTTCCTCCGGA-3′
SEQ ID NO.8 jd6093b-3′primer:
5′-AAGCTCGGGAAACCGGA-3′

The Semi-structured N40 pool and primers described in Table 2 were synthesized using an ABI Expedite 8909 synthesizer and deprotected using standard methods. The pool was then PolyPac purified. The pool was quantified, and large-scale PCR was performed to achieve a 3×10¹⁵ RNA molecule pool diversity. The PCR product was in vitro transcribed for six hours using 2′-fluoro modified UTP and CTP nucleotides (Durascribe Kit). Following transcription, the RNA pool was EtOH precipitated, and then gel purified. The gel slices were then electro-eluted, EtOH precipitated, and DNase treated. The final pool concentration was 24.18 uM (7.28×10¹⁵ total RNA molecules), containing enough for two and a half selection pools.

TABLE 3
Type I doped pool (jd10386a).

a. Pool template (jd10386a).

SEQ ID No.9 jd10386a:
(residues in the sequence preceded by a percent
symbol ‘%’ are 85% WT (i.e. the designated residue)
and 15% mutated (i.e. 15% likely to be any of the
other three possible residues))
5′-GGGAGACAAGAATAAACG%G%G%A%G%C%C%C%T%T
%C%G%C%A%C%G%A%A%A%G%T%G%C%C%A%C%T%A
%T%G%C%T%C%CTTCGACAGGAGGCTCACAA-3′
b. Pool primers (jd10388a/jd10381C).
SEQ ID NO.10 jd10388a-5′primer:
5′-TAATACGACTCACTATAGGGAGACAAGAATAAACG-3′
SEQ ID NO.11 jd10381c-3′primer:
5′-TTGTGAGCCTCCTGTCGAA-3′

The type I doped pool template described in Table 3 was synthesized using an ABI Expedite 8909 synthesizer and deprotected in-house using standard methods. The pool template was then PolyPac purified. The pool primers were synthesized by IDT. The pool was amplified by large-scale PCR to achieve a 5×10¹³ RNA molecule pool diversity. The PCR product was then in vitro transcribed overnight using 2′-fluoro modified UTP and CTP nucleotides (Durascribe Kit). Following transcription, the RNA pool was EtOH precipitated, DNase treated, and then gel purified. The gel slices were then passive eluted and EtOH precipitated. The final pool concentration was 41.5 uM. The pool was then tested for its ability to be reverse-transcribed and PCR amplified.

TABLE 4
Type II doped pool (jd10386b)

| | |
|---|---|
| a. Pool template (jd10386b). | +TL,1/32 |
| SEQ ID NO. 12 jd10386b | |
| (residues in the sequence preceded by a | |
| percent symbol ‘%’ are 85% WT | |
| (i.e. the designated residue) and 15% mutated | |
| (i.e. 15% likely to be any of the other three | |
| possible residues)) | |
| 5′-GGGAGACAAGAATAAACGG%G%A%G%C%C%C%A%C%C | |
| %C%G%A%C%G%A%A%A%G%T%C%G%C%C%C%A%A%G%C | |
| %T%C%CTTCGACAGGAGGCTCACAA-3′ | |
| b. Pool primers (jd10388a/jd10381c). | |
| SEQ ID No. 13 jd10388a-5′primer: | |
| 5′-TAATACGACTCACTATAGGGAGACAAGAATAAACG-3′ | |
| SEQ ID No. 14 jd10381c-3′primer: | |
| 5′-TTGTGAGCCTCCTGTCGAA-3′ | |

The type II doped pool template described in Table 4 was synthesized using an ABI Expedite 8909 synthesizer and deprotected using standard methods. The pool template was then PolyPac purified. The pool primers were synthesized by IDT. The pool was amplified by large-scale PCR to achieve a 5×10¹³ RNA molecule pool diversity. The PCR product was then in vitro transcribed overnight using 2′-fluoro modified UTP and CTP nucleotides. Following transcription, the RNA pool was EtOH precipitated, DNase treated, and then gel purified. The gel slices were then passive eluted and EtOH precipitated. The final pool concentration was 41.5 uM. The pool was then tested for its ability to be reverse-transcribed and PCRed.

TABLE 5
Type III doped pooi (jd10381a).

a. Pool template (jd10381a)
SEQ ID No. 15 jd10381a:
(residues in the sequence preceded by a percent
symbol ‘%’ are 85% WT
(i.e. the designated residue) and 15% mutated
(i.e. 15% likely to be any of the other
three possible residues))
5′-GGGAGACAAGAATAAACG%G%G%A%G%C%A%G%C%A
%C%C%G%A%A%A%G%G%T%G%G%C%A%A%G%T%C%G
%T%T%G%C%T%C%CTTCGACAGGAGGCTCAC-3′
b. Pool primers (jd10388a/jd10381c).
SEQ ID No. 16 jd10388a- 5′primer:
5′-TAATACGACTCACTATAGGGAGACAAGAATAAACG-3′
SEQ ID No. 17 jd10381c- 3′primer:
5′-TTGTGAGCCTCCTGTCGAA-3′

The type III doped pool template described in Table 5 was synthesized using an ABI Expedite 8909 synthesizer and deprotected using standard methods. The pool template was then PolyPac purified. The pool primers were synthesized by IDT. The pool was amplified by large-scale PCR to achieve a 5×10¹³ RNA molecule pool diversity. The PCR product was then in vitro transcribed overnight using 2′-fluoro modified UTP and CTP nucleotides. Following transcription, the RNA pool was EtOH precipitated, DNase treated, and then gel purified. The gel slices were then passive eluted and EtOH precipitated. The final pool concentration was 41.5 uM. The pool was then tested for its ability to be reverse-transcribed and PCRed.

### Example 2

### Plate-Based gp41 Aptamer Selection Against N36 Peptide Targets

A. Peptide Targets.

Several gp41 peptide selection targets were used during the selection and clone screening process as described below and in Table 6. Positive selections were performed against the gp41 N-terminal peptide. Negative selections were performed against the GCN4 peptide. A single mutant N-terminal peptide was used in the clone screening process to select for clones that had the greatest discrimination in binding. The peptides were synthesized using standard methods and were resuspended in 100% DMSO.

TABLE 6
gp41 Peptide Targets

A. N-terminal (N17) gp41 peptide (jd60127a).
SEQ ID No. 18 jd60127a:
(N-terminus)-biotin-GKG-RMKQIEDKIEEIESKQKKIENEIARIKK-LLQLTVWGIKQLQARIL-NH₂-(C-terminus)
B. GCN4 peptide (jd60127b).
SEQ ID No. 19 jd60127b:
(N-terminus)-biotin-GKG-RMKQIEDKIEEIESKQKKIENEIARIKK-NH₂-(C-terminus)
C. Single mutant N-terminal (N17) gp41 peptide (jd29a).
SEQ ID No. 20 jd10330a:
(N-terminus)-biotin-GKG-RMKQIEDKIEEIESKQKKIENEIARIKK-LLQLTVWWIKQLQARIL-NH₂(C-terminus)

B. Selection Protocol.

In the original plate-based selections against N-terminal gp41 peptide, NeutrAvidin plates were used to immobilize the biotinylated N-terminal gp41 peptide (jd60127a) to the plate surface. The Yang N40 (jd6054a) and the Semi-Structured N40 (jd6093e) naive pools (with a starting diversity of 2×10¹⁴ RNA molecules) were used to select for RNA molecules that bind the N-terminal gp41 peptide. In the positive selection, 1 uM positive peptide (jd60127a) was used. SHMCK buffer (pH 7.4, Hepes 20 mM, NaCl 120 mM, KCl 5 mM, CaCl₂ 1 mM, MgCl₂ 1 mM) was used as the binding buffer in this plate-based selection. This selection was concluded after eleven rounds. The protocol for selection Rounds 2 through 11 is described in Table 7.

TABLE 7
Protocol for Rounds 2 through 11 (see Round Notes for exceptions):

| | |
|---|---|
| 1. | Incubate 60 ul of 1 uM peptide stock and 6 ul of 10X SHMCK for |
| | 1 hr at 25° C. (RT) with shaking (jd60127b for negative and |
| | jd60127a for positive selection wells). Run plate washer 5 cycles |
| | (SHMCK w/Tween) |
| 2. | Incubate half of the transcription product from the previous round in |
| | 50 ul reaction vol. (1X SHMCK) for 1 hr at RT. (Neg. Selection- |
| | only after Round 5) |
| 3. | After neg. selection, transfer RNA to positive selection wells. Add |
| | 0.1 mg/mL tRNA and salmon sperm DNA to each well. Incubate at |
| | RT for 1 hr. |
| 4. | Remove reaction vol. and wash 4X with 200 ul 1X SHMCK |
| 5. | Add 75 ul RT Mix w/ThermoScript to positive selection wells and |
| | incubate at 65° C. for 1 hr. |
| 6. | Add 75 ul RT product to 175 ul PCR mix w/Taq and run PCR. Check |
| | after 15 cycles. |
| 7. | EtOH precipitate 50 ul PCR product and add 2′-F TXN Mix |
| | w/PPi and mutant T7. Incubate O/N at 37° C. |

Notes.
Round 2. Used one half of transcription product to go into Round 2.
Round 4. Increased the amount of wash cycles to 8 × 200 uL of 1X SHMCK.
Round 5. Instead of adding all of the RT product (75 uL) to the PCR, now adding 15 uL RT product to 100 uL PCR mix. Still EtOH precipitate 50 uL for the transcription reaction.
Round 6. Added the GCN4 peptide (jd60127b) to the negative selections.
Round 8. Instead of adding half of transcription product, now adding 1/10 of transcription product (5 uL) into Round 8. Increased the wash cycles to 4 × 200 uL 1X SHMCK. Wait 2 min. Wash 4 × 200 uL 1X SHMCK. Wait 2 min. Wash 2 × 200 uL 1X SHMCK.
Round 10. To increase the stringency, added 10 uM NeutrAvidin and 10 uM GCN4 peptide (jd60127b) to the positive selection reaction (in addition to the 1 uM N-terminal gp41 peptide (jd60127a)).

C. Aptamers.

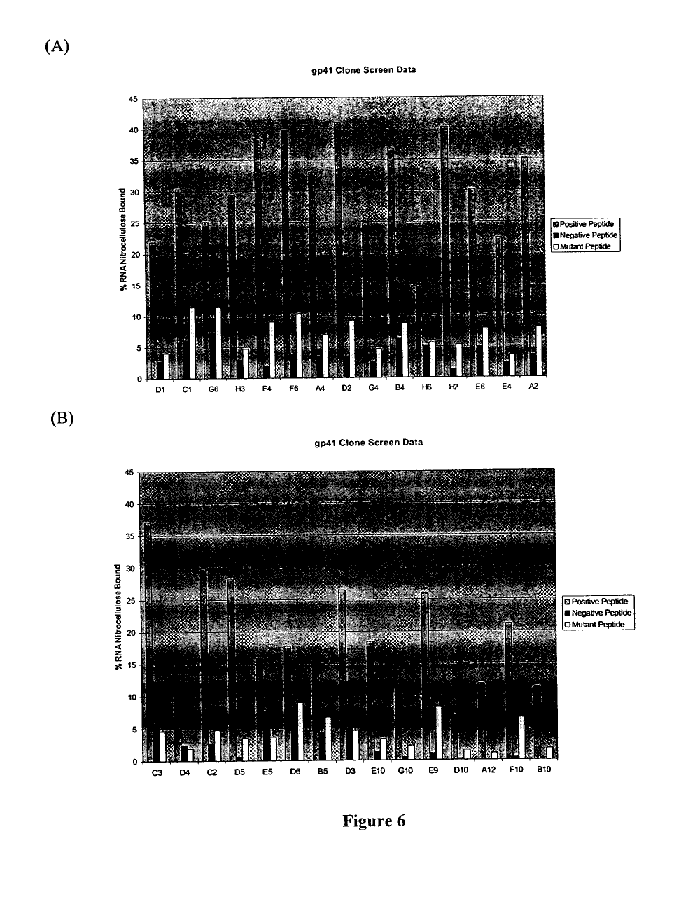

The plate-based selection for gp41 N-terminal peptide-dependent binders went through eleven rounds of selection. After the eleventh round, the binding properties of the pools were examined. Both the jd6054a and jd6093e selected pools bound to the positive peptide with a nanomolar K_{D} (130-140 nM). When the pools were tested for binding to the negative and the mutant peptides, they showed much lower binding; exhibiting a high binding specificity for the positive peptide. Since the pools showed a high specificity of binding after eleven rounds of selection, they were then sequenced (see Tables 8 and 9 below) and individual clones tested for their ability to bind to each of the three peptides (see FIGS. 6 (A) and (B)). Those clones that showed the greatest amount of specificity for the positive peptide were chosen and their K_{D}s were determined. To the extent tested, none of these aptamers exhibited an ability to block fusion in cell/cell fusion assays in which cells expressing HIV-1 envelope glycoprotein are mixed with cells expressing CD4 and coreceptor.

TABLE 8
N40Pool R11 original selection

SEQ ID No. 21>AMX(27).A1_D01_07
GGGAGACAAGAATAAACGCTCAATTATTCACAATCTGTCGAGCTAGTTTAACTAGCCAAGTCG-TTCGACAGGAGGCTCACAACAGGC
SEQ ID No. 21>AMX(27).ALB02_04
GGGAGACAAGAATAAACGCTCAATTATTCACAATCTGTCGAGCTAGTTTAACTAGCCAAGTCG-TTCGACAGGAGGCTCACAACAGGC
SEQ ID No. 21>AMX(27).A1_C06_06
GGGAGACAAGAATAAACGCTCAATTATTCACAATCTGTCGAGCTAGTTTAACTAGCCAAGTCG-TTCGACAGGAGGCTCACAACAGGC
SEQ ID No. 21>AMX(27).A1_F05_11
GGGAGACAAGAATAAACGCTCAATTATTCACAATCTGTCGAGCTAGTTTAACTAGCCAAGTCG-TTCGACAGGAGGCTCACAACAGGC
SEQ ID No. 22>AMX(27).A1_C01_05
GGGAGACAAGAATAAACGCTCAAGCCCAAGGACAGATTAGTCCCCACCCTTGTAACGTTCTTA-TTCGACAGGAGGCTCACAACAGGC
SEQ ID No. 22>AMX(27).A1_B06_04
GGGAGACAAGAATAAACGCTCAAGCCCAAGGACAGATTAGTCCCCACCCTTGTAACGTTCTTA-TTCGACAGGAGGCTCACAACAGGC
SEQ ID No. 22>AMX(27).A1_HO5_15
GGGAGACAAGAATAAACGCTCAAGCCCAAGGACAGATTAGTCCCCACCCTTGTAACGTTCTTA-TTCGACAGGAGGCTCACAACAGGC
SEQ ID No. 22>AMX(27).A1_A03_01
GGGAGACAAGAATAAACGCTCAAGCCCAAGGACAGATTAGTCCCCACCCTTGTAACGTTCTTA-TTCGACAGGAGGCTCACAACAGGC
SEQ ID No. 22>AMX(27).A1_A06_02
GGGAGACAAGAATAAACGCTCAAGCCCAAGGACAGATTAGTCCCCACCCTTGTAACGTTCTTA-TTCGACAGGAGGCTCACAACAGGC
SEQ ID No. 23>AMX(27).A1_G06_14
GGGAGACAAGAATAAACGCTCAAGCCCAAGGACAGATTAGTCCCCACCCTTGTAACGTTCTTA-TTCGACAGGAGGCTCACAACAGGC
SEQ ID No. 24>AMX(27).A1_H03_15
GGGAGACAAGAATAAACGCTCAATTATTCNNGATCTGTCGAGCTAGTTTNACTAGCCAAGTCG-TTCGACAGGAGGCTCACAACAGGC
SEQ ID No. 25>AMX(27).A1_F04_12
GGGAGACAAGAATAAACGCTCAAAGGGCGGTCCATAGTCGAGTCAGAATAACTGACCAAGTCG-TTCGACAGGAGGCTCACAACAGGC
SEQ ID No. 25>AMX(27).A1_C04_06
GGGAGACAAGAATAAACGCTCAAAGGGCGGTCCATAGTCGAGTCAGAATAACTGACCAAGTCG-TTCGACAGGAGGCTCACAACAGGC
SEQ ID No. 26>AMX(27).A1_F06_12
GGGAGACAAGAATAAACGCTCAATGAGTTCCGGTATGTCGAGCTGTGATAAACAGCCAAGTCG-TTCGACAGGAGGCTCACAACAGGC
SEQ ID No. 27>AMX(27).A1_A04_02
GGGAGACAAGAATAAACGCTCAATGAGGATCCTAGCGAGCCAGTGTAGTAACTGACCAAGTCG-TTCGACAGGAGGCTCACAACAGGC
SEQ ID No. 28>AMX(27).A1_D02_08
GGGAGACAAGAATAAACGCTCAAAATCAGAGCCATGTCGAAACCCCATCATGGGGTCAAGTCGTTTCGACAGGAGGCTCACAACAGGC
SEQ ID No. 29>AMX(27).A1_G04_14
GGGAGACAAGAATAAACGCTCAAAGCTTCGGAGATCCGAAGCCAAGTCGTTTTGGGATGATTA-TTCGACAGGAGGCTCACAACAGGC
SEQ ID No. 30>AMX(27).A1_B04_04
GGGAGACAAGAATAAACGCTCAACGTTGTGGAGACTGGCTAATTTATGAGTCGAATATATTGC-TTCGACAGGAGGCTCACAACAGGC
SEQ ID No. 31>AMX(27).A1_H06_16
GGGAGACAAGAATAAACGCTCAAAAGCTGTGGAGTGCTGTCGAGCTTAATCTAAGCCAAGTCG-TTCGACAGGAGGCTCACAACAGGC
SEQ ID No. 32>AMX(27).A1_H02_16
GGGAGACAAGAATAAACGCTCAAGTCTGCAGTGACCTGGCTGATGATAAGTCGAATGTAAGGC-TTCGACAGGAGGCTCACAACAGGC
SEQ ID No. 33>AMX(27).A1_F03_11
GGGAGACAAGAATAAACGCTCAAGTCTGCAGTGACCTGGCTGATGATAAGTCGAATGTAAGGC-TTCGACAGGAGGCTCACAACAAGC
SEQ ID No. 34>AMX(27).A1_E06_10
GGGAGACAAGAATAAACGCTGAATGAAATCCAGACGTTGGACACTACGGCGGCAGTTA------TTCGACAGGAGGCTCACAACAGGC
SEQ ID No. 34>AMX(27).A1_G02_14
GGGAGACAAGAATAAACGCTCAATGAAATCCAGACGTTGGACACTACGGCGGCAGTTA------TTCGACAGGAGGCTCACAACAGGC
SEQ ID No. 35>AMX(27).A1_E04_10
GGGAGACAAGAATAAACGCTCAATTCCGGAGCATACGCTGCATAAGTTTGCGGTACCATTTTA-TTCGACAGGAGGCTCACAACAGGC
SEQ ID No. 36>AMX(27).A1_A02_02
GGGAGACAAGAATAAACGCTCAATACCCAGAGCGTCAATGGCAACAGATTGCCAGTATGTTTA-TTCGACAGGAGGCTCACAACAGGC
SEQ ID No. 37>AMX(27).A1_C03_05
GGGAGACAAGAATAAACGCTCAAAAGAGTAAATGTCGATCCTATTTATATATAGGACAAGTCG-TTCGACAGGAGGCTCACAACAGGC
SEQ ID No. 38>AMX(27).A1_D04_08
GGGAGACAAGAATAAACGCTCAACCGTTCGTCTATTTGACGTGCCTGCGTTTTTGATAA---A-TTCGACAGGAGGCTCACAACAGGC
SEQ ID No. 39>AMX(27).A1_C02_06
GGGAGACAAGAATAAACGCTCAAAAGAGTAAATGTCGATCCTATTTATACATAGGACAAGTCG-TTCGACAGGAGGCTCACAACAGGC
SEQ ID No. 40>N40 RD 11_D5
GGGAGACAAGAATAAACGCTCAAAGGGNGGTCCNTAGTCGAGTCTAGAATAANTGACCAAGTCTTCGACAGGAGGCTCACAACAGGC
SEQ ID No. 41>N40 RD 11_E5
GGGAGACAAGAATAAACGCTCAAGAGTCCCGATCGAATACTACCGAGACCCACTCGCCTTTTTTTCGACAGGAGGCTCACAACAGGC
SEQ ID No. 42>N40 RD 11_D6
GGGAGACAAGAATAAACGCTCAAGCCCAAGATACCTTCACTAGGTATCCCACCCTTAGGCTTATTCGACAGGAGGCTCACAACAGGC
SEQ ID No. 43>N40 RD 11_B5
GGGAGACAAGAATAAACGCTCAAGCCCAAGCATGGAATTATCAATGCCCACCCTTAAGCCGTATTCGACAGGAGGCTCACAACAGGC
SEQ ID No. 44>N40 RD 11_D3
GGGAGACAAGAATAAACGCTCAACNANCCCNGANCCTGNCTAATGATAAGNTCTAATCTNTTATTCGACAGGAGGCTCACAACAGGC
SEQ ID No. 45>N40 RD 11_H3
GGGAGACAAGAATAAACGCTCAATTATTCNNGATCTGTCGAGCTAGTTTNACTAGCCAAGTCGTTCGACAGGAGGCTCACAACAGGC

### Example 3

### Clone Minimization

An analysis of the individual clone sequences revealed three main families of gp41 aptamers referred to as type I, type II, and type III aptamers. These families were used to construct minimized clones. Briefly, sequence motifs are observed in multiple contexts allowing for design of minimized contructs. Monomeric, homo-dimeric, and hetero-dimeric forms of these aptamers were created and their K_{D}s and/or IC₅₀s determined. To the extent tested, none of these aptamers exhibited an ability to block fusion in cell/cell fusion assays in which cells expressing HIV-1 envelope glycoprotein are mixed with cells expressing CD4 and coreceptor.

A. Monomeric Forms.

The monomeric forms of the Type I, Type II, and Type III aptamers were named ARC217(SEQ ID No. 53), ARC218 (SEQ ID No. 54), and ARC219 (SEQ ID No. 55) respectively.

The monomeric forms of the Type I, Type II, and
Type III aptamers were named ARC217(SEQ ID No. 53),
ARC218 (SEQ ID No. 54), and ARC219 (SEQ ID No. 55)
respectively.

A. Monomeric Forms.

SEQ ID No. 53 (Type 1 = ARG217)
5′-GGAGCCCUUCGCAGGAAAGUGCCACUAUGCUCC-3′
SEQ ID No. 54 (Type 2 = ARC218)
5′-GGAGGCCAGCCGACGAAAGUCGCCCAAGCUCG-3′
SEQ ID No. 55 (Type 3 = ARC219)
5′-GGAGCAGCACCGAAAGGUGCCAAGUCGUUGCUCC-3′
B. Homo-dimeric forms.
SEQ ID No. 56 Type I Homo-dimer (ARG217.d):
5′-GGAGCCCUUCGCACGAAAGUGCCACUAUGCUCCUUCCUUCC
UUCCUUCUCGCCCUUCGCACGAAAGUGCCACUAUGCGAG-3′
SEQ ID No. 57 Type II Homo-dimer (ARC218.d):
5′-GGAGCCCACCCGACGAAAGUCGCCCAAGCUCCUUCCUUCCUUCC
UUCUCGCCCACCCGACGAAAGUCGCCCAAGCGAG -3′
SEQ ID No. 58 Type III Homo-dimer (ARC219.d):
5′-GGAGCAGCACCGAAAGGUGCCAAGUCGUUGCUCCUUCCUUCC
UUCCUUCUCGCAGCACCGAAAGGUGCCAAGUCGUUGCGAG-3′
C. Hetero-dimeric forms.
SEQ ID No. 59 Type III-I Hetero-Dimer (ARC219.d31):
5′-GGAGCAGCACCGAAAGGUGCCAAGUCGUUGCUCCUUCCUUCC
UUCCUUCUCGCCCUUCGCACGAAAGUGCCACUAUGCGAG-3′
SEQ ID No. 60 Type I-III Hetero-Dimer (ARC217.d13):
5′-GGAGCCCUUCGCACGAAAGUGCCACUAUGCUCCUUCCUUCC
UUCCUUCUCGCAGCACCGAAAGGUGCCAAGUCGUUGCGAG-3′
SEQ ID No. 61 Type III-II Hetero-Dimer (ARC2 19.d32):
5′-GGAGCAGCACCGAAAGGUGCCAAGUCGUUGCUCCUUCCUUCC
UUCCUUCUCGCCCACCCGACGAAAGUCGCCCAAGCGAG-3′
SEQ ID No. 62 Type II-III Hetero-Dimer (ARC218.d23):
5′-GGAGCCCACCCGACGAAAGUCGCCCAAGCUCCUUCCUUCC
UUCCUUCUCGCAGCACCGAAAGGUGCCAAGUCGUUGCGAG-3′
SEQ ID No. 63 Type I-II Hetero-Dimer (ARC217.d12):
5′-GGAGCCCUUCGCACGAAAGUGCCACUAUGCUCCUUCCUUCC
UUCCUUCUCGCCCACCCGACGAAAGUCGCCCAAGCGAG-3′
SEQ ID No. 64 Type II-I Hetero-Dimer (ARC218.d21):
5′-GGAGCCCACCCGACGAAAGUCGCCCAAGCUCCUUCCUUCC
UUCCUUCUCGCCCUUCGCACGAAAGUGCCACUAUGCGAG-3′

### Example 4

### Plate-Based Doped Re-Selection Against N36 Peptide Targets

NeutrAvidin plates were used to immobilize the biotinylated N-terminal gp41 peptide (jd60127a) to the plate surface. The three doped pools: type I (jd10386a), type II (jd10386b), and type III (jd10381a) (with a starting diversity of 5×10¹³ RNA molecules) were used to select for RNA molecules that bind the N-terminal gp41 peptide. In the negative selection, 1 uM negative peptide (jd60127b) was used, and in the positive selection, 1 uM positive peptide (jd60127a) was used. SHMCK buffer was used as the binding buffer in this plate-based selection. These selections were concluded after five rounds and individual clones sequenced (See Tables 10, 11 and 12). The selection for Rounds 2 through 5 were unchanged as for the original selection as described above.

TABLE 10
Round 5 Type I (jd10386a) Pool.

SEQ ID No. 65>gp41type1_70B1
GGGAGACAAGAATAAACGCTCAATTATTCACAATCTGTCGAGCTAGT
TTACTAGCCAGTCGTTCGACAGGAGGCTCACAA
SEQ ID No. 65>gp41type1_70G2
GGGAGACAAGAATAAACGCTCAATTATTCACAATCTGTCGAGCTAGT
TTACTAGCCAGTCGTTCGACAGGAGGCTCACAA
SEQ ID No. 65>gp41type1_70G3
GGGAGACAAGAATAAACGCTCAATTATTCACAATCTGTCGAGCTAGT
TTACTAGCCAGTCGTTCGACAGGAGGCTCACAA
SEQ ID No. 66>gp41type1_70A2
GGGAGACAAGAATAAACGCTCAATTATTCACAATCTGTCGAGCTAGT
TTACTAGCCAGTCGTTCGACAGGAGGCTCACAA
SEQ ID No. 65>gp41type1_70F1
GGGAGACAAGAATAAACGCTCAATTATTCACAATCTGTCGAGCTAGT
TTACTAGCCAGTCGTTCGACAGGAGGCTCACAA
SEQ ID No. 65>gp41type1_70E3
GGGAGACAAGAATAAACGCTCAATTATTCACAATCTGTCGAGCTAGT
TTACTAGCCAGTCGTTCGACAGGAGGCTCACAA
SEQ ID No. 67>gp41type1_70F4
GGGAGACAAGAATAAACGCTCAATTATTCACAATCTGTCGAGCTAGT
TTACTAGCCAGTCGTTCGACAGGAGGCTCACAA
SEQ ID No. 65>gp41type1_70E5
GGGAGACAAGAATAAACGCTCAATTATTCACAATCTGTCGAGCTAGT
TTACTAGCCAGTCGTTCGACAGGAGGCTCACAA
SEQ ID No. 68>gp41type1_70E4
GGGAGACAAGAATAAACGCTCAATTATTCACAATCTGTCGAGCTAGT
TTACTAGCCAGTCGTTCGACAGGAGGCTCACAA
SEQ ID No. 69>gp41type1_70A6
GGGAGACAAGAATAAACGCTCAATTATTCACAATCTGTCGAGCTAGT
TTACTAGCCAGTCGTTCGACAGGAGGCTCACAA
SEQ ID No. 70>gp41type1_70H2
GGGAGACAAGAATAAACGCTCAATTATTCACAATCTGTCGAGCTAGT
TTACTAGCCAGTCGTTCGACAGGAGGCTCACAA
SEQ ID No. 70>gp41type1_70A1
GGGAGACAAGAATAAACGCTCAATTATTCACAATCTGTCGAGCTAGT
TTACTAGCCAGTCGTTCGACAGGAGGCTCACAA
SEQ ID No. 70>gp41type1_70C1
GGGAGACAAGAATAAACGCTCAATTATTCACAATCTGTCGAGCTAGT
TTACTAGCCAGTCGTTCGACAGGAGGCTCACAA
SEQ ID No. 71>gp41type1_70B2
GGGAGACAAGAATAAACGCTCAATTATTCACAATCTGTCGAGCTAGT
TTACTAGCCAGTCGTTCGACAGGAGGCTCACAA
SEQ ID No. 72>gp41type1_70D5
GGGAGACAAGAATAAACGCTCAATTATTCACAATCTGTCGAGCTAGT
TTACTAGCCAGTCGTTCGACAGGAGGCTCACAA
SEQ ID No. 73>gp41type1_70D2
GGGAGACAAGAATAAACGCTCAATTATTCACAATCTGTCGAGCTAGT
TNAACTAGCCCAGNTNNTNGACAGGAGGNTCACAA
SEQ ID No. 74>gp41type1_70G6
GGGAGACAAGAATAAACGCTCAACATGGGTCATCTGTGTCGAGTTGTA
ACAACAACCAAGTCGTTCGACAGGAGGCTCACAA
SEQ ID No. 75>gp41type1_70A3
GGGAGACAAGAATAAACGCTCAACATGGGTCATCTGGGTCGAGTTGTA
ACAACAACCAAGTCGTTCGACAGGAGGCTCACAA
SEQ ID No. 76>gp41type1_70C4
GGGAGACAAGAATAAACGCTCAAACATTGGGCACTGTCGAGTTCGTA
TTCACGGACCAAGTCGTTCGACAGGAGGCTCACAA
SEQ ID No. 77>gp41type1_70G1
GGGAGACAAGAATAAACGCTCAAGGTTGGAGTTTAGCTGTCGAGTACGT
GATGTACCAAGTCGTTCGACAGGAGGCTCACAA
SEQ ID No. 78>gp41type1_70E1
GGGAGACAAGAATAAACGCTCAAGTCTGCAGTGACCTGGCTGATGATAA
GTCGAATTTATTGCTTCGACAGGAGGCTCACAA
SEQ ID No. 79>gp41type1_70A4
GGGAGACAAGAATAAACGCTCAAGAGCAACAGTCGAGTCAAG
TCCACTTGACAAGTCGTTCGACAGGAGGCTCACAA
SEQ ID No. 80>gp41type1_70F3
GGGAGACAAGAATAAACGCTCAAGTCTGCAGTGACCTGGCTGATGATAA
GTCGAATGTAAGGCTTCGACAGGAGGCTCACAA
SEQ ID No. 80>gp41type1_70C6
GGGAGACAAGAATAAACGCTCAAGTCTGCAGTGACCTGGCTGATGATAA
GTCGAATGTAAGGCTTCGACAGGAGGCTCACAA
SEQ ID No. 80>gp41type1_70G5
GGGAGACAAGAATAAACGCTCAAGTCTGCAGTGACCTGGCTGATGATAA
GTCGAATGTAAGGCTTCGACAGGAGGCTCACAA
SEQ ID No. 81>gp41type1_70D6
GGGAGACAAGAATAAACGCTCAAGTCTGCAGTGACCTGGCTGATGATAA
GTCGAATGTAAGGCTTCGACAGGAGGCTCACAA
SEQ ID No. 82>gp41type1_70B5
GGGAGACAAGAATAAACGCTCAAGTCTGCAGTGACCTGGCTGATGATAA
GTCGAATGTAAGGCTTCGACAGGAGGCTCACAA
SEQ ID No. 83>gp41type1_70H5
GGGAGACAAGAATAAACGCTCAAGTCTGCAGTGACCTGGCTGATGATAA
GTCGAATGTAAGGCTTCGACAGGAGGCTCACAA
SEQ ID No. 83>gp41type1_70F6
GGGAGACAAGAATAAACGCTCAAGTCTGCAGTGACCTGGCTGATGATAA
GTCGAATGTAAGGCTTCGACAGGAGGCTCACAA
SEQ ID No. 84>gp41type1_70A5
GGGAGACAAGAATAAACGCTCAAGTCTGCAGTGACCTGGCTGATGATAA
GTCGAATGTAAGGCTTCGACAGGAGGCTCACAA
SEQ ID No. 85>gp41type1_70E2
GGGAGACAAGAATAAACGCTCAATCGTGGCTTTCTATCGAGTCCATA
TCGATGGACCAAGTCGTTCGACAGGAGGCTCACAA
SEQ ID No. 86>gp41type1_70D4
GGGAGACAAGAATAAACGCTCAAAGGGCGGTCCATAGTCGAGTCAGA
ATAACTGACCAAGTCGTTCGACAGGAGGCTCACAA
SEQ ID No. 87>gp41type1_70G4
GGGAGGCAAGAATAAACGCTTAAGTCTGCAGTGACCTGGCTAATGATAA
GTCGAATGTAAGGCTTCGACAGGAGGCTCACAA
SEQ ID No. 88>gp41type1_70H4
GGGAGACAAGAATAANCGCTCAAAATAGTGAGAAATGTCGATATCTC
GAGTAAAACTCGCCCATTCGACAGGAGGCTCACAA
SEQ ID No. 89>gp41type1_70C2
GGGAGACAAGAATAAACGCTCAAAATAGTGAGAAATGTCGATATCTC
GAGTAAAACTCGCCCATTCGACAGGAGGCTCACAA
SEQ ID No. 90>gp41type1_70H3
GGGAGACAAGAATAAACGCTCAAAAGAGTAAATGTCGATCCTATTTA
TACATAGGACAAGTCGTTCGACAGGAGGCTCACAA
SEQ ID No. 91>gp41type1_70D3
GGGAGACAAGAATAAACGCTCAAAAGAGTAAATGTCGATCCTATTTA
TACATAGGACAAGTCGTTCGACAGGAGGCTCACAA
SEQ ID No. 92>gp41type1_70F2
GGGAGACAAGAATAAACGCTCAAAAGAGTAAATGTCGATCCTATTTA
TACATAGGACAAGTCGTTCGACAGGAGGCTCACAA
SEQ ID No. 93>gp41type1_70B6
GGGAGACNAGAATAAACGCTCAAAACAGTGAGAAATGTCGATATCTC
GAGTAAAACTCGCCCATTCNACAGGAGGCTCACAA
SEQ ID No. 94>gp41type1_70B4
GGGAGACAAGAATAAACGCTCAAAATAGTGAGAGATGCCGATATCTC
GAGTAAAGCTCGCCCATTCGACAGGAGGCTCACAA
SEQ ID No. 95>gp41type1_70F5
GGGAGACAAGAATAAACGCCCAATGAAATCCAGACGTTGGACA
CTACGGCGGCAGTTATTCGACAGGAGGCTCACAA
SEQ ID No. 96>gp41type1_70H1
GGGAGACAAGAATAAACGCTCAAGCCCAAGGACAGATTAGTCCCCAC
CCTTGTAACGTTCTTATTCGACAGGAGGCTCACAA
SEQ ID No. 96>gp41type1_70E6
GGGAGACAAGAATAAACGCTCAAGCCCAAGGACAGATTAGTCCCCAC
CCTTGTAACGTTCTTATTCGACAGGAGGCTCACAA
SEQ ID No. 97>gp41type1_70D1
GGGAGACAAGAATAAACGCTCAAGCCCAAGATACCTTCACTAGGTAT
CCCACCCTTAGGCTTATTCGACAGGAGGCTCACAA

### Example 5

### Modified and Truncated Synthetic Clones

After doped re-selection, the type III aptamer (ARC219) was chosen to be truncated and modified. ARC219 was chosen because it exhibits both a high binding affinity and the greatest specificity of binding between the positive and mutant peptides.

A. ARC219 Based Synthetic Clone Sequences.

Following are synthetic sequences (see Table 13, aptamers ARC220 through ARC221.83 (jd132-3p)) that were made by truncating and/or modifying ARC219. In some cases, gp41aptamer K_{D}s and/or IC₅₀s were determined. To the extent tested, none of these aptamers exhibited an ability to block fusion in cell/cell fusion assays in which cells expressing HIV-1 envelope glycoprotein are mixed with cells expressing CD4 and coreceptor. In the sequences below, the symbol “[idT]” represents inverted thymidine.

TABLE 13
PEG Stabilized Aptamers

SEQ ID No. 152 ARC220 type III with idT
GGAGCAGCACCGAAAGGUGCCAAGUCGUUGCUCC[idT]
SEQ ID Nos. 153 & 154 ARC221 ARG220 with PEG linker
5′-GGAGCAGCACC-3′(SEQ ID No. 153)-PEG-5′
GGUGCCAAGUCGUUGCUCC[idT]-3′ (SEQ ID No. 154)
SEQ ID Nos. 153 & 154 ARC221.a all 2′OMe stem
5′-GGAGCAGCACC-3′(SEQ ID No. 153)-PEG-5′-GGUGCCAAGUCGUUGCUCC[idT]-3′
(SEQ ID No. 155)
SEQ ID Nos. 153 & 154 ARC221.b selected 2′OMe stem
5′-GGAGCAGCACC-3′(SEQ ID No. 153)-PEG-5′-GGUGCCAAGUCGUUGCUCC[idT]-3′
(SEQ ID No. 154)
SEQ ID Nos. 153 & 154 ARC221.1 full length R-2′OMe stem
5′-GGAGCAGCACC-3′(SEQ ID No. 153)-PEG-5′-GGUGCCAAGUCGUUGCUCC[idT]-3′
(SEQ ID No. 154)
SEQ ID Nos. 239 & 155 ARC221.2 R-2′OMe stem (−1,−1)
5′-GAGCAGCAC-3′ (SEQ ID No. 239)-PEG-5′-GUGCCAAGUCGUUGCUC[idT]-3′
(SEQ ID No. 155)
SEQ ID Nos. 240 & 156 ARC221.3 R-2′OMe stem (−2,−1)
5′-AGCAGCAC-3′ (SEQ ID No. 240)-PEG-5′-GUGCCAAGUCGUUGCU[idT]-3′
(SEQ ID No. 156)
SEQ ID Nos. 241 & 157 ARC221.4 R-2′OMe stem (−1,−2)
5′-GAGCAGCA-3′ (SEQ ID No. 241)-PEG-5′-UGCCAAGUCGUUGCUC[idT]-3′
(SEQ ID No. 157)
SEQ ID Nos. 242 & 158 ARC221.5 R-2′OMe stem (−2,−2)
5′-AGCAGCA-3′ (SEQ ID No. 242)-PEG-5′-UGCCAAGUCGUUGCU[idT]-3′
(SEQ ID No. 158)
SEQ ID Nos. 153 & 154 ARC22 1.6 full length all-R-2′OMe
5′-GGAGCAGCACC-3′ (SEQ ID No. 153)-PEG-5′-GGUGCCAAGUCGUUGUCC[idT]-3′
(SEQ ID No. 154)
SEQ ID Nos. 153 & 154 ARC221.7 full length Y-2′OMe stem
5′-GGUGCCAAGUCGUUGCUCC[idT]-3′
(SEQ ID No. 154)
SEQ ID Nos. 239 & 155 ARC221.8 Y-2′OMe stem (−1,−1)
5′-GAGCAGCAC-3′ (SEQ ID No. 239)-PEG-5′GUGCCAAGUCGUUGCUC[idT]-3′
(SEQ ID No. 155)
SEQ ID Nos. 240 & 156 ARC221.9 Y-2′OMe stem (−2,−1)
5′-AGCAGCAC-3′ (SEQ ID No. 240)-PEG-5′-GUGCCAAGUCGUUGCU[idT]-3′
(SEQ ID No. 156)
SEQ ID Nos. 241 & 157 ARC221.10 Y-2′OMe stem (−1,−2)
5′-GAGCAGCA-3′ (SEQ ID No. 24)-PEG-5′-UGCCAAGUCGUUGCUC[idT]-3′
(SEQ ID No. 157)
SEQ ID Nos. 242 & 158 ARC221.11 Y−2′OMe stem (−2,−2)
5′-AGCAGCA-3′ (SEQ ID No. 242)-PEG-5′-UGCCAAGUCGUUGCU[idT]-3′
(SEQ ID No. 158)
SEQ ID Nos. 159 & 154 ARC221.12 full length all-Y−2′OMe
5′-GGAG+E,usn CAGCACC-3′ (SEQ ID No. 159)-PEG-5′-GGUGCCAAGUCGUUGCUCC[idT]-3′
(SEQ ID No. 154)
SEQ ID Nos. 153 & 154 ARC221.13 full length R-DNA stem
5′-{double overscore (GGAG)}C{double overscore (AG)}C{double overscore (A)}CC-3′ (SEQ ID No. 153)-PEG-5′-{double overscore (GG)}U{double overscore (G)}CCAAGUCGUU{double overscore (G)}CUCC[idT]-3′
(SEQ ID No. 154)
SEQ ID Nos. 239 & 155 ARC221.14 R-DNA stem (−1,−1)
5′-{double overscore (GAG)}C{double overscore (AG)}C{double overscore (A)}C-3′ (SEQ ID No. 239)-PEG-5′-{double overscore (G)}U{double overscore (G)}CCAAGUCGUU{double overscore (G)}CUC[idT]-3′
(SEQ ID No. 155)
SEQ ID Nos. 240 & 156 ARC221.15 R-DNA stem (−2,−1)
5′-{double overscore (AG)}C{double overscore (AG)}C{double overscore (A)}C-3′ (SEQ ID No. 240)-PEG-5′-{double overscore (G)}U{double overscore (G)}CCAAGUCGUU{double overscore (G)}CU[idT]-3′
(SEQ ID No. 156)
SEQ ID Nos. 241 & 157 ARC221.16 R-DNA stem (−1,−2)
5′-{double overscore (GAG)}C{double overscore (AG)}C{double overscore (A)}-3′ (SEQ ID No. 241)-PEG-5′-U{double overscore (G)}CCAAGUCGUU{double overscore (G)}CUC[idT]-3′
(SEQ ID No. 157)
SEQ ID Nos. 242 & 158 ARC221.17 R-DNA stem (−2,−2)
5′-AGCAGCA-3′ (SEQ ID No. 242)-PEG-5′-U{double overscore (G)}CCAAGUCGUU{double overscore (G)}CU[idT]-3′
(SEQ ID No. 158)
SEQ ID Nos. 153 & 154 ARC221.18 full length all-R-DNA
5′-{double overscore (GGAG)}C{double overscore (AG)}C{double overscore (A)}CC-3′ (SEQ ID No. 153)-PEG-5′-{double overscore (GG)}U{double overscore (G)}CC{double overscore (AAG)}UC{double overscore (G)}UU{double overscore (G)}CUCC[idT]-3′
(SEQ ID No. 154)
SEQ ID Nos. 153 & 160 ARC221.19 full length R-2′OMe/Y-DNA stem
5′-GGAGCAG{double overscore (CACC)}-3′ (SEQ ID No. 153)-PEG-5′-GG{double overscore (T)}G{double overscore (C)}CAAGUCGU{double overscore (T)}G{double overscore (GCTCC)}[idT]-3′
(SEQ ID No. 160)
SEQ ID Nos. 239 & 161 ARC22l.20 R-2′OMe/Y-DNA stem (−1,−1)
5′-GAG{double overscore (C)}AG{double overscore (C)}A{double overscore (C)}-3′ (SEQ ID No. 239)-PEG-5′-G{double overscore (T)}G{double overscore (C)}CAAGUCGU{double overscore (T)}G{double overscore (CTC)}[idT]-3′
(SEQ ID No. 161)
SEQ ID Nos. 240 & 162 ARC221.21 R-2′OMe/Y-DNA stem (−2,−1)
5′-AG{double overscore (C)}AG{double overscore (C)}A{double overscore (C)}-3′ (SEQ ID No. 240)-PEG-5′-G{double overscore (T)}G{double overscore (C)}CAAGUCGU{double overscore (T)}G{double overscore (CT)}[idT]-3′
(SEQ ID No. 162)
SEQ ID Nos. 241 & 163 ARC221.22 R-2′OMe/Y-DNA stem (−1,−2)
5′-GAG{double overscore (C)}AG{double overscore (C)}A-3′ (SEQ ID No. 241)-PEG-5′-{double overscore (T)}G{double overscore (C)}CAAGUCGU{double overscore (T)}G{double overscore (CTC)}[idT]-3′
(SEQ ID No. 163)
SEQ ID Nos. 242 & 164 ARC22l.23 R-2′OMe/Y-DNA stem (−2,−2)
5′-AG{double overscore (C)}AG{double overscore (C)}A-3′ (SEQ ID No. 242)-PEG-5′-{double overscore (T)}G{double overscore (C)}CAAGUCGU{double overscore (T)}G{double overscore (CT)}[idT]-3′
(SEQ ID No. 164)
SEQ ID Nos. 153 & 165 ARC221.24 full length all-R-2′OMe/Y-DNA
5′-GGAG{double overscore (C)}AG{double overscore (C)}A{double overscore (CC)}-3′ (SEQ ID No. 153)-PEG-5′-GG{double overscore (T)}G{double overscore (CC)}AAG{double overscore (TC)}G{double overscore (CTCC)}[idT]-3′
(SEQ ID No. 165)
SEQ ID Nos. 153 & 154 ARC221.25 full length R-DNA stem
5′-{double overscore (GGAG)}C{double overscore (AG)}C{double overscore (A)}CC-3′ (SEQ ID No. 153)-PEG-5′-{double overscore (GG)}U{double overscore (G)}CCAAGUCGUU{double overscore (G)}CUCC[idT]-3′
(SEQ ID No. 154)
SEQ ID Nos. 239 & 155 ARC221.26 R-DNA stem (−1,−1)
5′-{double overscore (GAG)}C{double overscore (AG)}C{double overscore (A)}C-3′ (SEQ ID No. 239)-PEG-5′-{double overscore (G)}U{double overscore (G)}CCAAGUCGUU{double overscore (G)}CUC[idT]-3′
(SEQ ID No. 155)
SEQ ID Nos. 240 & 156 ARC221.27 R-DNA stem (−2,−1)
5′-{double overscore (AG)}C{double overscore (AG)}C{double overscore (A)}C-3′ (SEQ ID No. 240)-PEG-5′-{double overscore (G)}U{double overscore (G)}CCAAGUCGUU{double overscore (G)}CUC[idT]-3′
(SEQ ID No. 156)
SEQ ID Nos. 241 & 157 ARC221.28 R-DNA stem (−1,−2)
5′-{double overscore (GAG)}C{double overscore (AG)}C{double overscore (A)}-3′ (SEQ ID No. 241)-PEG-5′-U{double overscore (G)}CCAAGUCGUU{double overscore (G)}CUC[idT]-3′
(SEQ ID No. 157)
SEQ ID Nos. 242 & 158 ARC221.29 R-DNA stem (−2,−2)
5′-{double overscore (AG)}C{double overscore (AG)}C{double overscore (A)}-3′ (SEQ ID No. 242)-PEG-5′-U{double overscore (G)}CCAAGUCGUU{double overscore (G)}CU[idT]-3′
(SEQ ID No. 158)
SEQ ID Nos. 153 & 154 ARC221.30 A full length all-R-DNA
5′-{double overscore (GGAG)}C{double overscore (AG)}C{double overscore (A)}CC-3′ (SEQ ID No. 153)-PEG-5′-{double overscore (GG)}U{double overscore (G)}CC{double overscore (AAG)}UC{double overscore (G)}UU{double overscore (G)}CUCC[idT]-3′
(SEQ ID No. 154)
SEQ ID Nos. 240 & 156 ARC221.30. B full length all-R-DNA
5′-AGCAGCAC-3′ (SEQ ID No. 240)-PEG-5′-GUGCCAAGUCGUUGCU-3′
(SEQ ID No. 156)
SEQ ID Nos. 241 & 157 ARC221.30. C full length all-R-DNA
5′-GAGCAGCA-3′ (SEQ ID No. 241)-PEG-5′-UGCCAAGUCGUUGCUC-3′
(SEQ ID No. 157)
SEQ ID Nos. 239 & 155 ARC221.30. D full length all-R-DNA
5′-GAGCAGCAC-3′ (SEQ ID No. 239)-PEG-5′-GUGCCAAGUCGUUGCUC-3′
(SEQ ID No. 155)
SEQ ID Nos. 242 & 158 ARC221.30. E full length all-R-DNA
5′-AGCAGCA-3′ (SEQ ID No. 242)-PEG-5′-UGCCAAGUCGUUGCU-3′
(SEQ ID No. 158)
SEQ ID Nos. 166 & 167 ARC221.31 all 2′OMe stem full length
5′-mGmGmCmGmCmAmGmCmGmCmC-3′ (SEQ ID No. 166)-PEG-5′-mGmGmCmGmCfCAAGfUfCGfUmUmGmCmGmCmC[idT]-3′
(SEQ ID No. 167)
SEQ ID Nos. 243 & 168 ARC221.32 all 2′OMe stem (−1,−1)
5′-mGmCmGmCmAmGmCmGmC-3′ (SEQ ID No. 243)-PEG-5′-mGmCmGmCfCAAGfUfCGfUmUmGmCmGmC[idT]-3′
(SEQ ID No. 168)
SEQ ID Nos. 244 & 169 ARC221.33 all 2′OMe stem (−2,−2)
5′-mCmGmCmAmGmCmGmC-3′ (SEQ ID No. 244)-PEG-5′-mGmCmGmCfCAAGfUfCGfUmUmGmCmG[idT]-3′
(SEQ ID No. 169)
SEQ ID Nos. 161 & 170 ARC221.34 all 2′OMe stem full length plus 2′OMe purine/2′-F
pyrimidine in loop
5′-mGmGmCmGmCmAmGmCmGmCmC-3′ (SEQ ID No. 161)-PEG-5′-mGmGmCmGmCfCmAmAmGfUfCmGfUmUmGmCmGmCmC[idT]-3′
(SEQ ID No. 170)
SEQ ID Nos. 161 & 171 ARC221.35 all 2′OMe stem full length plus ribo-purine/2′-OMe
pyrimidine in loop
5′-mGmGmCmGmCmAmGmCmGmCmC-3′ (SEQ ID No. 161)-PEG-5′-mGmGmCmGmCmCAAGmUmCGmUmUmGmCmGmCmC[idT]-3′
(SEQ ID No. 171)
SEQ ID Nos. 161 & 172 ARC221.36 all 2′-OMe full length
5′-mGmGmCmGmCmAmGmCmGmCmC-3′ (SEQ ID No. 161)-PEG-5′-mGmGmCmGmCmCmAmAmGmUmCmGmUmUmGmCmGmCmC[idT]-3′
(SEQ ID No. 172)
SEQ ID Nos. 173 & 174 ARC221.37 deoxy-purine/2′OMe pyrimidine stem full length
5′-dGdGmCdGmCdAdGmCdGmCmC-3′ (SEQ ID No. 173)-PEG-5′-dGdGmCdGmCfCAAGfUfCGfUmUdGmCdGmCmC[idT]-3′
(SEQ ID No. 174)
SEQ ID Nos. 243 & 175 ARC221.38 deoxy-purine/2′OMe pyrimidine stem (−1,−1)
5′-dGdGmCdGmCdAdGmCdGmCmC-3′ (SEQ ID No. 243)-PEG-5′-dGmCdGmCfCAAGfUfCGfUmUdGmCdGmC[idT]-3′
(SEQ ID No. 175)
SEQ ID Nos. 244 & 176 ARC221.39 deoxy-purine/2′OMe pyrimidine stem (−2,−2)
5′-mCdGmCdAdGmCdGmC-3′ (SEQ ID No. 244)-PEG-5′-dGmCdGmCfCAAGfUfCGfUmUdGmCdG[idT]-3′
(SEQ ID No. 176)
SEQ ID Nos. 177 & 178 ARC221.40 deoxy-purine/2′OMe pyrimidine stem full length plus
deoxy-purine/2′-F pyrimidine in loop
5′-dGdGmCdGmCdAdGmCdGmCmC-3′ (SEQ ID No. 177)-PEG-5′-dGdGmCdGmCfCdAdAdGfUfCdGfUmUdGmCdGmCmC[idT]-3′
(SEQ ID No. 178)
SEQ ID Nos. 177 & 179 ARC221.41 deoxy-purine/2′OMe pyrimidine stem full length all
deoxy-purine/2′-OMe pyrimidine
5′-dGdGmCdGmCdAdGmCdGmCmC-3′ (SEQ ID No. 177)-PEG-5′-dGdGmCdGmCmCdAdAdGmUmCdGmUmUdGmCdGmCmC[idT]-3′
(SEQ ID No. 179)
SEQ ID Nos. 245 & 180 ARC221.42 (jd132−2a) delete bp 3-32/bp 9-18
5′-rGrGrGfCrArGfCfCfC-3′ (SEQ ID No. 245)-PEG-5′=rGrGrGfCfCrArArGfUfCrGfUfUrGfCfCfC[idT]-3′
(SEQ ID No. 180)
SEQ ID Nos. 246 & 181 ARC221.43 (jd132-2b) delete bp 3-32/bp 4-31/ bp 9-18
5′-rGrGfCrArGfCfCfC-3′ (SEQ ID No. 246)-PEG-5′-rGrGrGfCfCrArArGfUfCrGfUfUrGfCfC[idT]-3′
(SEQ ID No. 181)
SEQ ID Nos. 247 & 182 ARC221.44 (jd132-2c) delete bp 3-32/ bp 9-18/bp 10-17
5′-rGrGrGfCrArGfCfC-3′ (SEQ ID No. 247)-PEG-5′-rGrGfCfCrArArGfUfCrGfUfUrGfCfCfC[idT]-3′
(SEQ ID No. 182)
SEQ ID Nos. 248 & 183 ARC221.45 (jd132-2d) delete bp 3-32/bp 4-31/bp 9−18/ bp 10-17
5′-rGrGfCrArGfCfC-3′ (SEQ ID No. 248)-PEG-5′-rGrGfCfCrArArGfUfCrGfUfUrGfCfC[idT]-3′
(SEQ ID No. 183)
SEQ ID Nos. 249 & 184 ARC221.46 (jd132-2e) delete bp 3-32/bp 4-31/bp 6−29/ bp 9-18
5′-rGrGfCrGfCfCfC-3′ (SEQ ID No. 249)-PEG-5′-rGrGrGfCfCrArArGfUfCrGfUrGfCfC[idT]-3′
(SEQ ID No. 184)
SEQ ID Nos. 250 & 185 ARC221.47 (jd132-2f) delete bp 3-32/bp 4-31/bp 6−29/ bp 9-18/
bp 10-17
5′-rGrGfCrGfCfC-3′ (SEQ ID No. 250)-PEG-5′-rGrGfCfCrArArGfUfCrGfUrGfCfC[idT]-3′
(SEQ ID No. 185)
SEQ ID Nos. 186 & 187 ARC221.48 (jd132-2g) sense sequence
5′-rGrGrArGfCrArGfCrAfCfC-3′ (SEQ ID No. 186)-PEG-5′-rGrGfUrGfCfCrArArGfUfCrGfUfUrGfCfUfCfC[idT]-3′
(SEQ ID No. 187)
SEQ ID Nos. 186 & 188 ARC221.49 (jd132-2h) Pyrimidine-to-OMe: residue 5
5′-rGrGrArGmCrArGfCrAfCfC-3′ (SEQ ID No. 186)-PEG-5′-rGrGfUrGfCfCrArArGfUfCrGfUfUrGfCfUfCfC[idT]-3′
(SEQ ID No. 188)
SEQ ID Nos. 186 & 189 ARC221.50 (jd132-2i) Pyrimidine-to-OMe: residue 29
5′-rGrGrArGfCrArGfCrAfCfC-3′ (SEQ ID No. 186)-PEG-5′-rGrGfUrGfCfCrArArGfUfCrGfUfCfC[idT]-3′
(SEQ ID No. 189)
SEQ ID Nos. 186 & 190 ARC221.51 (jd132-2j) Pyrimidine-to-OMe: residue 31
5′-rGrGrArGfCrArGfCrAfCfC-3′ (SEQ ID No. 186)-PEG-5′-rGrGfUrGfCfCrArArGfUfCrGfUfUrGmCfUfCfC[idT]-3′
(SEQ ID No. 190)
SEQ ID Nos. 186 & 191 ARCZ21.52 (jd132-2k) Pyrimidine-to-OMe: residue 32
5′-rGrGrArGfCrArGfCrAfCfC-3′ (SEQ ID No. 186)-PEG-5′-rGrGfUrGfCfCrArArGfUfCrGfUfUrGfCmUfCfC[idT]-3′
(SEQ ID No. 191)
SEQ ID Nos. 186 & 192 ARC221.53 (jd132-21) Pyrimidine-to-OMe: residue 33
5′-rGrGrArGfCrArGfCrAfCfC-3′ (SEQ ID No. 186)-PEG-5′-rGrGfUrGfCfCrArArGfUfCrGfUfUrGfCfUmCfC[idT]-3′
(SEQ ID No. 192)
SEQ ID Nos. 186 & 193 ARC221.54 (jd132-2m) Pyrimidine-to-OMe: residue 34
5′-rGrGrArGfCrArGfCrAfCfC-3′ (SEQ ID No. 196)-PEG-5′-rGrGfUrGfCfCrArArGfUfCrGfUfUrGfCfUfCmC[idT]-3′
(SEQ ID No. 193)
SEQ ID Nos. 188 & 194 ARC221.55 (jd132-2n) Pyrimidine-to-OMe: combined
5′-rGrGrArGmCrArGfCrAfCfC-3′ (SEQ ID No. 188)-PEG-5′-rGrGfUrGfCfCrArArGfUfCrGfUmUrGmCmUmCmC[idT]-3′
(SEQ ID No. 194)
SEQ ID Nos. 195 & 187 ARC221.56 (jd132-2o) Purine-to-DNA: residue 1
5′-dGrGrArGfCrArGfCrAfCfC-3′ (SEQ ID No. 195)-PEG-5′-rGrGfUrGfCfCrArArGfUfCrGfUfUrGfCfUfCfC[idT]-3′
(SEQ ID No. 187)
SEQ ID Nos. 196 & 187 ARC221.57 (jd132-2p) Purine-to-DNA: residue 2
5′-rGdGrArGfCrArGfCrAfCfC-3′ (SEQ ID No. 196)-PEG-5′-rGrGfUrGfCfCrArArGfUfCrGfUfUrGfCfUfCfC[idT]-3′
(SEQ ID No. 187)
SEQ ID Nos. 197 & 187 ARC221.58 (jd132-2q) Purine-to-DNA: residue 3
5′-rGrGdArGfCrArGfCrAfCfC-3′ (SEQ ID No. 197)-PEG-5′-rGrGfUrGfCfCrArArGfUfCrGfUfUrGfCfUfCfC[idT]-3′
(SEQ ID No. 187)
SEQ ID Nos. 198 & 187 ARC221.59 (jd132-2r) Purine-to-DNA: residue 4
5′-rGrGrAdGfCrArGfCrAfCfC-3′ (SEQ ID No. 198)-PEG-5′-rGrGfUrGfCfCrArArGfUfCrGfUfUrGfCfUfCfC[idT]-3′
(SEQ ID No. 187)
SEQ ID Nos. 199 & 187 ARC221.60 (jd132-2s) Purine-to-DNA: residue 6
5′-rGrGrArGfCdArGfCrAfCfC-3′ (SEQ ID No. 199)-PEG-5′-rGrGfUrGfCfCrArArGfUfCrGfUfUrGfCfUfCfC[idT]-3′
(SEQ ID No. 187)
SEQ ID Nos. 200 & 201 ARC221.61 (jd132-2t) Purine-to-DNA: residue 30
5′-rGrGrArGfCrArGfCrAfCfC-3′ (SEQ ID No. 200)-PEG-5′-rGrGfUrGfCfCrArArGfUfCrGfUfUdGfCfUfCfC[idT]-3′
(SEQ ID No. 201)
SEQ ID Nos. 202 & 201 ARC221.62 (jd132-2u) Purine-to-DNA: combined
5′-dGdGdAdGfCdArGfCrAfCfC-3′ (SEQ ID No. 202)-PEG-5′-rGrGfUrGfCfCrArArGfUfCrGfUfUdGfCfUfCfC[idT]-3′
(SEQ ID No. 201)
SEQ ID Nos. 203 & 187 ARC221.63 (jd132-2v) Pyrimidine-to-OMe: residue 8
5′-rGrGrArGfCrArGmCrAfCfC-3′ (SEQ ID No. 203)-PEG-5′-rGrGfUrGfCfCrArArGfUfCrGfUfUrGfCfUfCfC[idT]-3′
(SEQ ID No. 187)
SEQ ID Nos. 204 & 187 ARC22l.64 (jd132-2w) Pyrimidine-to-OMe: residue 10
5′-rGrGrArGfCrArGfCrAmCfC-3′ (SEQ ID No. 204)--PEG-5′-rGrGfUrGfCfCrArArGfUfCrGfUfUrGfCfUfCfC[idT]-3′
(SEQ ID No. 187)-
SEQ ID Nos. 205 & 187 ARC221.65 (jd132-2x) Pyrimidine-to-OMe: residue 11
5′-rGrGrArGfCrArGfCrAfCmC-3′ (SEQ ID No. 205)--PEG-5′-rGrGfUrGfCfCrArArGfUfCrGfUfUrGfCfUfCfC[idT]-3′
(SEQ ID No. 187)-
SEQ ID Nos. 200 & 206 ARC22I.66 (jd132-2y) Pyrimidine-to-OMe: residue 18
5′-rGrGrArGfCrArGfCrAfCfC-3′ (SEQ ID No. 200)--PEG-5′-rGrGmUrGfCfCrArArGfUfCrGfUfUrGfCfUfCfC[idT]-3′
(SEQ ID No. 206)-
SEQ ID Nos. 200 & 207 ARC22I.67 (jd132-2z) Pyrimidine-to-OMe: residue 20
5′-rGrGrArGfCrArGfCrAfCfC-3′ (SEQ ID No. 200)--PEG-5′-rGrGfUrGmCfCrArArGfUfCrGfUfUrGfCfUfCfC[idT]-3′
(SEQ ID No. 207)-
SEQ ID Nos. 208 & 209 ARC221.68 (jd132-3a) Pyrimidine-to-OMe: combined
5′-rGrGrArGfCrArGmCrAmCmC-3′ (SEQ ID No. 208)-PEG-5′-rGrGmUrGmCfCrArArGfUfCrGfUfUrGfCfUfCfC[idT]-3′
(SEQ ID No. 209)-
SEQ ID Nos. 210 & 187 ARC22l.69 (jd132-3b) Purine-to-DNA: residue 7
5′-rGrGrArGfCrAdGfCrAfCfC-3′ (SEQ ID No. 210)-PEG-5′-rGrGfUrGfCfCrArArGfUfCrGfUfUrGfCfUfCfC[idT]-3′
(SEQ ID No. 187)
SEQ ID Nos. 211 & 187 ARC221.70 (jd132-3c) Purine-to-DNA: residue 9
5′-rGrGrArGfCrArGfCdAfCfC-3′ (SEQ ID No. 211)-PEG-5′-rGrGfUrGfCfCrArArGfUfCrGfUfUrGfCfUfCfC[idT]-3′
(SEQ ID No. 187)
SEQ ID Nos. 200 & 212 ARC22I.71 (jd132-3d) Purine-to-DNA: residue 16
5′-rGrGrArGfCrArGfCrAfCfC-3′ (SEQ ID No. 200)-PEG-5′-dGrGfUrGfCfCrArArGfUfCrGfUfUrGfCfUfCfC[idT]-3′
(SEQ ID No. 212)
SEQ ID Nos. 200 & 213 ARC221.72 (jd132-3e) Punne-to-DNA: residue 17
5′-rGrGrArGfCrArGfCrAfCfC-3′ (SEQ ID No. 200)-PEG-5′-rGdGfUrGfCfCrArArGfUfCrGfUfUrGfCfUfCfC[idT]-3′
(SEQ ID No. 213)
SEQ ID Nos. 200 & 214 ARC221.73 (jd132-3f) Purine-to-DNA: residue 19
5′-rGrGrArGfCrArGfCrAfCfC-3′ (SEQ ID No. 200)-PEG-5′-rGrGfUdGfCfCrArArGfUfCrGfUfUrGfCfUfCfC[idT]-3′
(SEQ ID No. 214)
SEQ ID Nos. 215 & 216 ARC221.74 (jd132-3g) Purine-to-DNA: combined
5′-rGrGrArGfCrAdGfCdAfCfC-3′ (SEQ ID No. 215)-PEG-5′-dGdGfUdGfCfCrArArGfUfCrGfUfUrGfCfUfCfC[idT]-3′
(SEQ ID No. 216)
SEQ ID Nos. 200 & 217 ARC221.75 (jd132-3h) any-to-DNA: residue 21
5′-rGrGrArGfCrArGfCrAfCfC-3′ (SEQ ID No. 200)-PEG-5′-rGrGfUrGfCdCrArArGfUfCrGfUfUrGfCfUfCfC[idT]-3′
(SEQ ID No. 217)
SEQ ID Nos. 200 & 218 ARC221.76 (jd132-3i) any-to-DNA: residue 22
5′-rGrGrArGfCrArGfCrAfCfC-3′ (SEQ ID No. 200)-PEG-5′-rGrGfUrGfCfCdArArGfUfCrGfUfUrGfCfUfCfC[idT]-3′
(SEQ ID No. 218)
SEQ ID Nos. 200 & 219 ARC221.77 (jd132-3j) any-to-DNA: residue 23
5′-rGrGrArGfCrArGfCrAfCfC-3′ (SEQ ID No. 200)-PEG-5′-rGrGfUrGfCfCrAdArGfUfCrGfUfUrGfCfUfCfC[idT]-3′
(SEQ ID No. 219)
SEQ ID Nos. 200 & 220 ARC221.78 (jd132-3k) any-to-DNA: residue 24
5′-rGrGrArGfCrArGfCrAfCfC-3′ (SEQ ID No. 200)-PEG-5′-rGrGfUrGfCfCrArAdGfUfCrGfUfUrGfCfUfCfC[idT]-3′
(SEQ ID No. 220)
SEQ ID Nos. 200 & 221 ARC221.79 (jd132-31) any-to-DNA: residue 25
5′-rGrGrArGfCrArGfCrAfCfC-3′ (SEQ ID No. 200)-PEG-5′-rGrGfUrGfCfCrArArGTtFCrGfUfUrGfCfUfCfC[idT]-3′
(SEQ ID No. 221)
SEQ ID Nos. 200 & 222 ARC221.80 (jd132-3m) any-to-DNA: residue 26
5′-rGrGrArGfCrArGfCrAfCfC-3′ (SEQ ID No. 200)-PEG-5′-rGrGfUrGfCfCrArArGfUdCrGfUfUrGfCfUfCfC[idT]-3′
(SEQ ID No. 222)
SEQ ID Nos. 200 & 223 ARC221.81 (jd132-3n) any-to-DNA: residue 27
5′-rGrGrArGfCrArGfCrAfCfC-3′ (SEQ ID No. 200)-PEG-5′-rGrGfUrGfCfCrArArGfUfCdGfUfUrGfCfUfCfC[idT]-3′
(SEQ ID No. 223)
SEQ ID Nos. 200 & 224 ARC221.82 (jd132-3o) any-to-DNA: residue 28
5′-rGrGrArGfCrArGfCrAfCfC-3′ (SEQ ID No. 200)-PEG-5′-rGrGfUrGfCfCrArArGfUfCrGTfUrGfCfUfCfC[idT]-3′
(SEQ ID No. 224)
SEQ ID Nos. 200 & 225 ARC221.83 (jd132-3p) any-to-DNA: combined
5′-rGrGrArGfCrArGfCrAfCfC-3′ (SEQ ID No. 200)-PEG-5′-rGrGfUrGfCdCdAdAdGTdCdGTfUrGfCfUfCfC[idT]-3′
(SEQ ID No. 225)

### Example 6

### gp41 Aptamer Selection with 2′F and 2′O-Me Modified Pools Against a Structurally Constrained C34: 2F5 Epitope Peptide

In addition to the selections for gp41 specific binding aptamers described above, selections were also performed to generate aptamers against HIV-1 gp41 C-terminal 2F5 epitope peptide, which consists of two peptide regions linked by diaminopropionic acid: jd103155a: Biotin-QNQQEKNEQELLE-(SEQ ID No. 234)—(diaminopropionic acid)-DKW-D₁₈-SLWNWF-CONH₂ (SEQ ID No. 235) with a lactam bridge between diaminopropionic acid and aspartic acid at position 18 producing a structural constraint, using two different pools, one containing 2′O-methyl- and the other containing 2′-fluoro-modified UTP and CTP.

The template, 5′ and 3′ primers for the 2′-O-methyl selections are described below. Pool templates include two oligonucleotides of defined sequence separated by a randomized region of 30 or 40 nucleotides in length (e.g., N30 or N40).

| | |
|---|---|
| SEQ ID Nos. 226 & 227: | |
| Pool Template (ARC 255) | |
| 5′-GGGAGAGGAGAGAACG-3′ | (SEQ ID No. 226) |
| -N30- 5′-CGGCTAGTCAGTCGCGATGCATG-3′ | (SEQ ID No. 227) |

SEQ ID No.228: 5′ Primer (PB.118.95.G)
5′-TAATACGACTCACTATAGGGAGAGGAGAGAACG-3′
SEQ ID No.229: 3′ Primer (PB118.95.H)
5′-CATGCATCGCGACTGACTAGCCG-3′.

### Example 7

### gp41 Aptamer Selection with 2′F and 2′OMe Modified Pools Against gp41 C34: 2F5 Epitope Peptide

Selections were also performed to generate aptamers against HIV-1 gp41C-terminal 2F5 peptide (SEQ ID No. 236: jd103155b:Biotin-QNQQEKNEQELLE-L-DKW-A-SLWNWF-CONH₂) using the 2′-OMe modified and 2′fluoro modified templates and primers of Example 6 to prepare the pools.

References cited above by author and year of publication are given their full citation below, and is each herein incorporated by reference in its entirety.

Broder, C. C., and Dimitrov, D. S. (1996) Pathobiology 64, 171-179.

Burton D R. (1997) “A vaccine for HIV type 1: the antibody perspective.” Proc Natl Acad Sci USA. 94: 10018-23.

Chan, D. C., Fass, D., Berger, J. M., and Kim, P. S. (1997), Cell 89, 263-273.

Chan, D. C., and Kim, P. S. (1998) Cell 93, 681-684.

Clerici, M., Barassi, C., Devito, C., Pastori, C., Piconi, S., Trabattoni, D., Longhi, R., Hinkula, J., Broliden, K., and Lopalco, J., “Serum IgA of HIV-exposed uninfected individuals inhibit HIV through recognition of a region within the α-helix of gp41” AIDS 2002, Vol 16, pp. 1731-1741.

Cole, J. L., and Garsky, V. M., “Thermodynamics of Peptide Inhibitor Binding to HIV-1 gp41” Biochemistry 2001, 40, pp.5633-5641.

Conley, A. J., Kessler, J. A., II, Boots, L. J., Tung, J.-S., Arnold, B. A., Keller, P. M., Shaw, A. R., and Emini, E. A (1994) PNAS 91, 3348-3352.

Dorn, J., Masciotra, S., Yang, C., Downing, R., Biryahwaho, B., Mastro, T. M., Nkengasong, J., Pieniazek, D., Rayfield, M., Hu, D. J., and Lal, R. B., “Analysis of Genetic Variability within the Immunodominant Epitopes of Envelope gp41 from Human Immundefieciecy Virus Type 1 (HIV-1_Group M and its impact on HIV-1 Antibody Detection.” J. of Clin. Microbiol., February 2000, Vol. 38 No.2, pp773-780.

D'Souza, M. P., and Harden, V. A. (1996) Nat. Med. 2, 1293-1300.

Eckert, D. M., Malashkevich, V. N., Hong, L. H., Carr, P. A., Kim, P. S. (1999) “Inhibiting HIV-1 entry: Discovery of D-peptide Inhibitors that target the gp41 Coiled-Coil Pocket” Cell, Vol. 99, 103-115.

Eckert, D. M., Kim, P. S. “Design of potent inhibitors of HIV-1 entry from the gp41 N-peptide region” PNAS 2001, Vol. 98, No.20 11187-11192.

Jiang, S., Lin, K., Strick, N., and Neurath, A. R. (1993) Nature 365, 113.

Judice, J. K., Tom, J. Y. K., Huang, W., Wrin, T., Vennari, J., Petropoulos, C. J., and McDowell, R. S. (1997) PNAS 94,13426- 13430.

Kilby, J. M., Hopkins, S., Venetta, T. M., DiMassimo, B., Cloud, G. A., Lee, J. Y., Alldredge, L., Hunter, E., Lambert, D., Bolognesi, D., Matthews, T., Johnson, M. R., Nowak, M. A., Shaw, G. M., and Saag, M. S. (1998) Nat. Med. 4, 1302-1307.

Kowalski M, Potz J, Basiripour L, Dorfman T, Goh W C, Terwilliger E, Dayton A, Rosen C, Haseltine W, Sodroski J. (1987) “Functional regions of the envelope glycoprotein of human immunodeficiency virus type 1.” Science. 237: 1351-5.

Lawless, M. K., Barney, S., Guthrie, K. I., Bucy, T. B., Petteway, S. R., and Merutka, G. (1996) Biochemistry 35, 13697-13708.

Lu M, Blacklow S C, Kim P S. (1995) “A trimeric structural domain of the HIV-1 transmembrane glycoprotein.” Nat Struct Biol. 2: 1075-82.

McGaughey G B, Citron M, Danzeisen R C, Freidinger R M, Garsky V M, Hurni W M, Joyce J G, Liang X, Miller M, Sjiver J, Bogusky M J (2003) “HIV-1 vaccine development: constrained peptide immunogens show improved binding to anti-HIV-1 gp41 MAb” Biochemistry. 42: 3214-23.

Muster, T., Steindl, F., Purtscher, M., Trkola, A., Klima, A., Himmler, G., Ruker, F., and Katinger, H. (1993) J. Virol. 67,6642-6647.

Poignard P, Saphire E O, Parren P W, Burton D R. (2001) “gp120: Biologic aspects of structural features.” Annu Rev Immunol. 19: 253-74.

Profy A T, Salinas P A, Eckler L I, Dunlop N M, Nara P L, Putney S D. (1990) “Epitopes recognized by the neutralizing antibodies of an HIV-1-infected individual.” J Immunol. 144: 4641-7.

Report on the Global HIV/AIDS Epidemic, XIV International Conference on AIDS, Barcelona, 7-12 Jul., 2002.

Root, M. J., Kay, M. S., and Kim, P. S. (2001) Science 291, 884-888.

Sackett, K. and Shai, Y., “The HIV-1 gp41 N-terminal Heptad Repeat Plays an Essential Role in Membrane Fusion” Biochemistry 2002, 41, 4678-4685.

Salzwedel, K., West, J. T., and Hunter, E. (1999) J. Virol. 73, 2469-2480.

Schibli, D. J., Montelaro, R. C., and Vogel, H. J. (2001) Biochemistry 40, 9570-9578.

Sia, S. K., Carr, P. A., Cochran, A. G., Malashkevich, V. N., Kim, P., “Short Constrained Peptides that Inhibit HIV-1 Entry” PNAS 2002, Vol.99, NO.23, pp.14664-14669.

Siebert, X., and Hummer, G., “Hydrophobicity Maps of the N-peptide Coiled Coil of HIV-1 gp41” Biocehmistry, 2002, Vol. 41, pp.2956-2961.

Singh, M., Berger, B., and Kim, P. S. (1999). LearnCoil-VMF: Computational evidence for coiled-coil-like motifs in many viral membrane fusion proteins. J. Mol. Biol. 290, 1031-1041.

Starcich B R, Hahn B H, Shaw G M, McNeely P D, Modrow S, Wolf H, Parks E S, Parks W P, Josephs S F, Gallo R C, et al. (1986) “Identification and characterization of conserved and variable regions in the envelope gene of HTLV-III/LAV, the retrovirus of AIDS.” Cell. 45: 637-48.

St. John Jones, P. L. Korte, T., Blumenthal, R., “Conformational Changes in Cell SurfaceHIV-1 Envelope Glycoproteins Are triggered by Cooperation between Cell Surface CD4 and Co-receptors” J. Bio. Chemistry 1998, Vol.273 No.1 pp. 404-409.

Suárez, T., Gallaher, W. R., Agirre, A., Goñi, F. M., and Nieva, J. L. (2000) J. Virol. 74, 8038-8047.

Tucker C E, Chen L S, Judkins M B, Farmer J A, Gill S G, Drolet D W (1999) “Detection and plasma pharmacokinetics of an anti-vascular endothelial growth factor oligonucleotide aptamer (NX1838) in rhesus monkeys.” J. Chromatography B. 732: 203-12.

Wild, C. T., Shugars, D. C., Greenwell, T. K., McDanal, C. B., and Matthews, T. J. (1994) PNAS 91, 9770-9774.

Wilkinson, D. (1996) Curr. Biol. 6, 1051-1053.

The invention having now been described by way of written description and examples, those of skill in the art will recognize that the invention can be practiced in a variety of embodiments and that the foregoing description and examples are for purposes of illustration and not limitation of the following claims.

What is claimed is:

1) An aptamer selected from the group consisting of SEQ ID No. 21, SEQ ID No. 22 through SEQ ID No. 151, and those aptamers listed in Table 13.

2) A therapeutic composition for treating subjects having HIV-infection comprising an aptamer selected from the group consisting of SEQ ID No. 21, SEQ ID No. 22 through SEQ ID No.151, and those aptamers listed in Table 13.

3) A method of treating HIV infection in a subject comprising the steps of administering to a subject a therapeutically effective amount of a composition comprising an aptamer selected from the group consisting of SEQ ID No. 21, SEQ ID No. 22 through SEQ ID No. 151, and those aptamers listed in Table 13 in a pharmaceutically acceptable form.

4) The method of claim 3, wherein said aptamer is complexed with a toxin moiety toxic to HIV particles or cells.

5) A method of diagnosing HIV infection in a sample comprising providing a sample from a subject suspected of HIV exposure, contacting the sample with an aptamer having a sequence selected from the group consisting of SEQ ID No. 21, SEQ ID No. 22 through SEQ ID No. 151, and those aptamers listed in Table 13, said aptamer complexed with a reporter moiety, wherein a signal is generated upon binding to an HIV target.

6) A method of treating HIV infection in a subject comprising the steps of administering to a subject a therapeutically effective amount of a composition comprising an aptamer that inhibits or blocks fusion of the viral and cell membranes.

7) The method of claim 6, wherein the aptamer blocks or inhibits the ability of gp41 to form a hairpin-like structure.


SEQUENCE LISTING (250 sequences)

SEQ ID NO: 1 - RNA, 22 nt, Artificial
  synthetic primer / aptamer
ggagacaagaauaaacgcucaa

SEQ ID NO: 2 - RNA, 24 nt, Artificial
  synthetic primer / aptamer
uucgacaggaggcucacaacaggc

SEQ ID NO: 3 - DNA, 40 bp, Artificial
  synthetic primer / aptamer
taatacgactcactatagggagacaagaataaacgctcaa

SEQ ID NO: 4 - DNA, 24 bp, Artificial
  synthetic primer / aptamer
gcctgttgtgagcctcctgtcgaa

SEQ ID NO: 5 - DNA, 16 bp, Artificial
  synthetic primer / aptamer
ggagccttcctccgga

SEQ ID NO: 6 - DNA, 17 bp, Artificial
  synthetic primer / aptamer
tccggtttcccgagctt

SEQ ID NO: 7 - DNA, 33 bp, Artificial
  synthetic primer / aptamer
taatacgactcactataggagccttcctccgga

SEQ ID NO: 8 - DNA, 17 bp, Artificial
  synthetic primer / aptamer
aagctcgggaaaccgga

SEQ ID NO: 9 - DNA, 70 bp, Artificial
  synthetic primer / aptamer
gggagacaagaataaacgggagcccttcgcacgaaagtgccactatgctccttcgacaggaggctcacaa

SEQ ID NO: 10 - DNA, 35 bp, Artificial
  synthetic primer / aptamer
taatacgactcactatagggagacaagaataaacg

SEQ ID NO: 11 - DNA, 19 bp, Artificial
  synthetic primer / aptamer
ttgtgagcctcctgtcgaa

SEQ ID NO: 12 - DNA, 69 bp, Artificial
  synthetic primer / aptamer
gggagacaagaataaacgggagcccacccgacgaaagtcgcccaagctccttcgacaggaggctcacaa

SEQ ID NO: 13 - DNA, 35 bp, Artificial; identical to SEQ ID NO: 10
  synthetic primer / aptamer

SEQ ID NO: 14 - DNA, 19 bp, Artificial
  synthetic primer / aptamer
ttgtgagcctcctgtcgaa

SEQ ID NO: 15 - DNA, 71 bp, Artificial
  synthetic primer / aptamer
gggagacaagaataaacgggagcagcaccgaaaggtgccaagtcgttgctccttcgacaggaggctcacaa

SEQ ID NO: 16 - DNA, 35 bp, Artificial; identical to SEQ ID NO: 10
  synthetic primer / aptamer

SEQ ID NO: 17 - DNA, 19 bp, Artificial
  synthetic primer / aptamer
ttgtgagcctcctgtcgaa

SEQ ID NO: 18 - protein, 48 aa, Artificial
  synthetic primer / aptamer
GKGRMKQIEDKIEEIESKQKKIENEIARIKKLLQLTVWGIKQLQARIL

SEQ ID NO: 19 - protein, 31 aa, Artificial
  synthetic primer / aptamer
GKGRMKQIEDKIEEIESKQKKIENEIARIKK

SEQ ID NO: 20 - protein, 48 aa, Artificial
  synthetic primer / aptamer
GKGRMKQIEDKIEEIESKQKKIENEIARIKKLLQLTVWWIKQLQARIL

SEQ ID NO: 21 - DNA, 87 bp, Artificial
  synthetic primer / aptamer
gggagacaagaataaacgctcaattattcacaatctgtcgagctagtttaactagccaagtcgttcgacaggaggctcacaacaggc

SEQ ID NO: 22 - DNA, 87 bp, Artificial
  synthetic primer / aptamer
gggagacaagaataaacgctcaagcccaaggacagattagtccccacccttgtaacgttcttattcgacaggaggctcacaacaggc

SEQ ID NO: 23 - DNA, 87 bp, Artificial
  synthetic primer / aptamer
gggagacaagaataaacgctcaagcccaaggacagactagtccccacccttgtaacgttcttattcgacaggaggctcacaacaggc

SEQ ID NO: 24 - DNA, 87 bp, Artificial
  synthetic primer / aptamer
gggagacaagaataaacgctcaattattcnngatctgtcgagctagtttnactagccaagtcgttcgacaggaggctcacaacaggc

SEQ ID NO: 25 - DNA, 87 bp, Artificial
  synthetic primer / aptamer
gggagacaagaataaacgctcaaagggcggtccatagtcgagtcagaataactgaccaagtcgttcgacaggaggctcacaacaggc

SEQ ID NO: 26 - DNA, 87 bp, Artificial
  synthetic primer / aptamer
gggagacaagaataaacgctcaatgagttccggtatgtcgagctgtgataaacagccaagtcgttcgacaggaggctcacaacaggc

SEQ ID NO: 27 - DNA, 87 bp, Artificial
  synthetic primer / aptamer
gggagacaagaataaacgctcaatgaggatcctagcgagccagtgtagtaactggccaagtcgttcgacaggaggctcacaacaggc

SEQ ID NO: 28 - DNA, 88 bp, Artificial
  synthetic primer / aptamer
gggagacaagaataaacgctcaaaatcagagccatgtcgaaaccccatcatggggtcaagtcgtttcgacaggaggctcacaacaggc

SEQ ID NO: 29 - DNA, 87 bp, Artificial
  synthetic primer / aptamer
gggagacaagaataaacgctcaaagcttcggagatccgaagccaagtcgttttgggatgattattcgacaggaggctcacaacaggc

SEQ ID NO: 30 - DNA, 87 bp, Artificial
  synthetic primer / aptamer
gggagacaagaataaacgctcaacgttgtggagactggctaatttatgagtcgaatatattgcttcgacaggaggctcacaacaggc

SEQ ID NO: 31 - DNA, 87 bp, Artificial
  synthetic primer / aptamer
gggagacaagaataaacgctcaaaagctgtggagtgctgtcgagcttaatctaagccaagtcgttcgacaggaggctcacaacaggc

SEQ ID NO: 32 - DNA, 87 bp, Artificial
  synthetic primer / aptamer
gggagacaagaataaacgctcaagtctgcagtgacctggctgatgataagtcgaatgtaaggcttcgacaggaggctcacaacaggc

SEQ ID NO: 33 - DNA, 87 bp, Artificial
  synthetic primer / aptamer
gggagacaagaataaacgctcaagtctgcagtgacctggctgatgataagtcgaatgtaaggcttcgacaggaggctcacaacaagc

SEQ ID NO: 34 - DNA, 82 bp, Artificial
  synthetic primer / aptamer
gggagacaagaataaacgctcaatgaaatccagacgttggacactacggcggcagttattcgacaggaggctcacaacaggc

SEQ ID NO: 35 - DNA, 87 bp, Artificial
  synthetic primer / aptamer
gggagacaagaataaacgctcaattccggagcatacgctgcataagtttgcggtaccattttattcgacaggaggctcacaacaggc

SEQ ID NO: 36 - DNA, 87 bp, Artificial
  synthetic primer / aptamer
gggagacaagaataaacgctcaatacccagagcgtcaatggcaacagattgccagtatgtttattcgacaggaggctcacaacaggc

SEQ ID NO: 37 - DNA, 87 bp, Artificial
  synthetic primer / aptamer
gggagacaagaataaacgctcaaaagagtaaatgtcgatcctatttatatataggacaagtcgttcgacaggaggctcacaacaggc

SEQ ID NO: 38 - DNA, 84 bp, Artificial
  synthetic primer / aptamer
gggagacaagaataaacgctcaaccgttcgtctatttgacgtgcctgcgtttttgataaattcgacaggaggctcacaacaggc

SEQ ID NO: 39 - DNA, 87 bp, Artificial
  synthetic primer / aptamer
gggagacaagaataaacgctcaaaagagtaaatgtcgatcctatttatacataggacaagtcgttcgacaggaggctcacaacaggc

SEQ ID NO: 40 - DNA, 87 bp, Artificial
  synthetic primer / aptamer
gggagacaagaataaacgctcaaagggnggtccntagtcgagtctagaataantgaccaagtcttcgacaggaggctcacaacaggc

SEQ ID NO: 41 - DNA, 87 bp, Artificial
  synthetic primer / aptamer
gggagacaagaataaacgctcaagagtcccgatcgaatactaccgagacccactcgcctttttttcgacaggaggctcacaacaggc

SEQ ID NO: 42 - DNA, 87 bp, Artificial
  synthetic primer / aptamer
gggagacaagaataaacgctcaagcccaagataccttcactaggtatcccacccttaggcttattcgacaggaggctcacaacaggc

SEQ ID NO: 43 - DNA, 87 bp, Artificial
  synthetic primer / aptamer
gggagacaagaataaacgctcaagcccaagcatggaattatcaatgcccacccttaagccgtattcgacaggaggctcacaacaggc

SEQ ID NO: 44 - DNA, 87 bp, Artificial
  synthetic primer / aptamer
gggagacaagaataaacgctcaacnancccngancctgnctaatgataagntctaatctnttattcgacaggaggctcacaacaggc

SEQ ID NO: 45 - DNA, 87 bp, Artificial; identical to SEQ ID NO: 24
  synthetic primer / aptamer

SEQ ID NO: 46 - DNA, 73 bp, Artificial
  synthetic primer / aptamer
ggagccttcctccggaaacctttaaccgccttcgcactattaagtgccactatcggtccggtttcccgagctt

SEQ ID NO: 47 - DNA, 73 bp, Artificial
  synthetic primer / aptamer
ggagccttcctccggaaacctttaaccgccttcgcactattaagtgccactaccggtccggtttcccgagctt

SEQ ID NO: 48 - DNA, 73 bp, Artificial
  synthetic primer / aptamer
ggagccttcctccggaaacttgtgaccaccctctgaatttttagttcaaagcccaatccggtttcccgagctt

SEQ ID NO: 49 - DNA, 73 bp, Artificial
  synthetic primer / aptamer
ggagccttcctccggagaccgaacaccctttgatcatttttcatggttagagcccatccggtttcccgagctt

SEQ ID NO: 50 - DNA, 73 bp, Artificial
  synthetic primer / aptamer
ggagccttcctccggaaattcgtgaccatcactacttcgtttagaaagtaccttcgtccggtttcccgagctt

SEQ ID NO: 51 - DNA, 73 bp, Artificial
  synthetic primer / aptamer
ggagccttcctccggaaatcgctgtagtccaaggaatttaataaaatttcccaccctccggtttcccgagctt

SEQ ID NO: 52 - DNA, 73 bp, Artificial
  synthetic primer / aptamer
ggagccttcctccggaatccggcctttaatagtagttagctcactactgtttaccatccggtttcccgagctt

SEQ ID NO: 53 - RNA, 33 nt, Artificial
  synthetic primer / aptamer
ggagcccuucgcacgaaagugccacuaugcucc

SEQ ID NO: 54 - RNA, 32 nt, Artificial
  synthetic primer / aptamer
ggagcccacccgacgaaagucgcccaagcucc

SEQ ID NO: 55 - RNA, 34 nt, Artificial
  synthetic primer / aptamer
ggagcagcaccgaaaggugccaagucguugcucc

SEQ ID NO: 56 - RNA, 80 nt, Artificial
  synthetic primer / aptamer
ggagcccuucgcacgaaagugccacuaugcuccuuccuuccuuccuucucgcccuucgcacgaaagugccacuaugcgag

SEQ ID NO: 57 - RNA, 78 nt, Artificial
  synthetic primer / aptamer
ggagcccacccgacgaaagucgcccaagcuccuuccuuccuuccuucucgcccacccgacgaaagucgcccaagcgag

SEQ ID NO: 58 - RNA, 82 nt, Artificial
  synthetic primer / aptamer
ggagcagcaccgaaaggugccaagucguugcuccuuccuuccuuccuucucgcagcaccgaaaggugccaagucguugcgag

SEQ ID NO: 59 - RNA, 81 nt, Artificial
  synthetic primer / aptamer
ggagcagcaccgaaaggugccaagucguugcuccuuccuuccuuccuucucgcccuucgcacgaaagugccacuaugcgag

SEQ ID NO: 60 - RNA, 81 nt, Artificial
  synthetic primer / aptamer
ggagcccuucgcacgaaagugccacuaugcuccuuccuuccuuccuucucgcagcaccgaaaggugccaagucguugcgag

SEQ ID NO: 61 - RNA, 80 nt, Artificial
  synthetic primer / aptamer
ggagcagcaccgaaaggugccaagucguugcuccuuccuuccuuccuucucgcccacccgacgaaagucgcccaagcgag

SEQ ID NO: 62 - RNA, 80 nt, Artificial
  synthetic primer / aptamer
ggagcccacccgacgaaagucgcccaagcuccuuccuuccuuccuucucgcagcaccgaaaggugccaagucguugcgag

SEQ ID NO: 63 - RNA, 79 nt, Artificial
  synthetic primer / aptamer
ggagcccuucgcacgaaagugccacuaugcuccuuccuuccuuccuucucgcccacccgacgaaagucgcccaagcgag

SEQ ID NO: 64 - RNA, 79 nt, Artificial
  synthetic primer / aptamer
ggagcccacccgacgaaagucgcccaagcuccuuccuuccuuccuucucgcccuucgcacgaaagugccacuaugcgag

SEQ ID NO: 65 - DNA, 82 bp, Artificial
  synthetic primer / aptamer
gggagacaagaataaacgctcaattattcacaatctgtcgagctagtttaactagccaagtcgttcgacaggaggctcacaa

SEQ ID NO: 66 - DNA, 82 bp, Artificial
  synthetic primer / aptamer
gggagacnagaataaacgctcaattattcacaatctgtcgagctagtttaactagccaagtcgttcgacaggaggctcacaa

SEQ ID NO: 67 - DNA, 82 bp, Artificial
  synthetic primer / aptamer
gggagacaagaagaaacgcccaattattcacaatctgtcgagctagtttaactagccaagtcgttcgacaggaggctcacaa

SEQ ID NO: 68 - DNA, 82 bp, Artificial
  synthetic primer / aptamer
gggagacaagaataaacgctcaattattcacgatctgtcgagctagtttaactagccaagtcgttcgacaggaggctcacaa

SEQ ID NO: 69 - DNA, 82 bp, Artificial
  synthetic primer / aptamer
gggagacacgaataaacgctcaattattcacgatctgtcgagctagtttaactagccaagtcgttcgacaggaggctcacaa

SEQ ID NO: 70 - DNA, 82 bp, Artificial; identical to SEQ ID NO: 68
  synthetic primer / aptamer

SEQ ID NO: 71 - DNA, 82 bp, Artificial
  synthetic primer / aptamer
gggagacaagaataaacgctcaattattcncgatctgtcgagctagtttaactagccaagtcgttngacaggaggctcacaa

SEQ ID NO: 72 - DNA, 82 bp, Artificial
  synthetic primer / aptamer
gggagacaagaatgaacgctcanttattcacaatctgtcgagctagtttaactagccaagtcgttcgacaggaggctcacaa

SEQ ID NO: 73 - DNA, 80 bp, Artificial
  synthetic primer / aptamer
gggacaagaataaacgctcaattnttcncaatctgtcgagctagttnaactagcccagntnntngacaggaggntcacaa

SEQ ID NO: 74 - DNA, 82 bp, Artificial
  synthetic primer / aptamer
gggagacaagaataaacgctcaacatgggtcatctgtgtcgagttgtaacaacaaccaagtcgttcgacaggaggctcacaa

SEQ ID NO: 75 - DNA, 82 bp, Artificial
  synthetic primer / aptamer
gggagacaagaataaacgctcaacatgggtcatctgggtcgagttgtaacaacaaccaagtcgttcgacaggaggctcacaa

SEQ ID NO: 76 - DNA, 82 bp, Artificial
  synthetic primer / aptamer
gggagacaagaataaacgctcaaacattgggcactgtcgagttcgtattcacggaccaagtcgttcgacaggaggctcacaa

SEQ ID NO: 77 - DNA, 82 bp, Artificial
  synthetic primer / aptamer
gggagacaagaataaacgctcaaggttggagtttagctgtcgagtacgtgatgtaccaagtcgttcgacaggaggctcacaa

SEQ ID NO: 78 - DNA, 82 bp, Artificial
  synthetic primer / aptamer
gggagacaagaataaacgctcaagtctgcagtgacctggctgatgataagtcgaatttattgcttcgacaggaggctcacaa

SEQ ID NO: 79 - DNA, 77 bp, Artificial
  synthetic primer / aptamer
gggagacaagaataaacgctcaagagcaacagtcgagtcaagtccacttgacaagtcgttcgacaggaggctcacaa

SEQ ID NO: 80 - DNA, 82 bp, Artificial
  synthetic primer / aptamer
gggagacaagaataaacgctcaagtctgcagtgacctggctgatgataagtcgaatgtaaggcttcgacaggaggctcacaa

SEQ ID NO: 81 - DNA, 82 bp, Artificial
  synthetic primer / aptamer
gggagacaagcataaacgcccaagtctgcagtgacctggctgatgataagtcgaatgtaaggcttcgacaggaggctcacaa

SEQ ID NO: 82 - DNA, 82 bp, Artificial
  synthetic primer / aptamer
gggagacaagaataaacgctcaagtctgcagtgacctggctaatgataagtcgaatgtaaggcttcgacaggaggctcacaa

SEQ ID NO: 83 - DNA, 82 bp, Artificial
  synthetic primer / aptamer
gggagacaagaataaacgctcaagtctgcagtgacctggctnatgataagtcgaatgtaaggcttcgacaggaggctcacaa

SEQ ID NO: 84 - DNA, 82 bp, Artificial
  synthetic primer / aptamer
gggagacaagaataaacgctcangtctgcagtgacctggctgatgataagtcgaatgtaaggcttcgacaggaggctcacaa

SEQ ID NO: 85 - DNA, 82 bp, Artificial
  synthetic primer / aptamer
gggagacaagaataaacgctcaatcgtggctttctatcgagtccatatcgatggaccaagtcgttcgacaggaggctcacaa

SEQ ID NO: 86 - DNA, 82 bp, Artificial
  synthetic primer / aptamer
gggagacaagaataaacgctcaaagggcggtccatagtcgagtcagaataactgaccaagtcgttcgacaggaggctcacaa

SEQ ID NO: 87 - DNA, 82 bp, Artificial
  synthetic primer / aptamer
gggaggcaagaataaacgcttaagtctgcagtgacctggctaatgataagtcgaatgtaaggcttcgacaggaggctcacaa

SEQ ID NO: 88 - DNA, 82 bp, Artificial
  synthetic primer / aptamer
gggagacaagaataancgctcaaaatagtgagaaatgtcgatatctcgagtaaaactcgcccattcgacaggaggctcacaa

SEQ ID NO: 89 - DNA, 82 bp, Artificial
  synthetic primer / aptamer
gggagacaagaataaacgctcaaaatagtgagaaatgtcgatatctcgagtaaaactcgcccattcgacaggaggctcacaa

SEQ ID NO: 90 - DNA, 82 bp, Artificial
  synthetic primer / aptamer
gggagacaagaataaacgctcaaaagagtaaatgtcgatcctatttatacataggacaagtcgttcgacaggaggctcacaa

SEQ ID NO: 91 - DNA, 82 bp, Artificial
  synthetic primer / aptamer
gggagacgagagtaaacnctcaaaagagtaaatgtcgatcctatttatacataggacaagtcgttcgacaggaggctcacaa

SEQ ID NO: 92 - DNA, 82 bp, Artificial
  synthetic primer / aptamer
gggagacaagaataaacgctcaaaagagtaaatgtcgatcctatttatatataggacaagtcgttcgacaggaggctcacaa

SEQ ID NO: 93 - DNA, 82 bp, Artificial
  synthetic primer / aptamer
gggagacnagaataaacgctcaaaacagtgagaaatgtcgatatctcgagtaaaactcgcccattcnacaggaggctcacaa

SEQ ID NO: 94 - DNA, 82 bp, Artificial
  synthetic primer / aptamer
gggagacaagaataaacgctcaaaatagtgagagatgccgatatctcgagtaaagctcgcccattcgacaggaggctcacaa

SEQ ID NO: 95 - DNA, 77 bp, Artificial
  synthetic primer / aptamer
gggagacaagaataaacgcccaatgaaatccagacgttggacactacggcggcagttattcgacaggaggctcacaa

SEQ ID NO: 96 - DNA, 82 bp, Artificial
  synthetic primer / aptamer
gggagacaagaataaacgctcaagcccaaggacagattagtccccacccttgtaacgttcttattcgacaggaggctcacaa

SEQ ID NO: 97 - DNA, 82 bp, Artificial
  synthetic primer / aptamer
gggagacaagaataaacgctcaagcccaagataccttcactaggtatcccacccttaggcttattcgacaggaggctcacaa

SEQ ID NO: 98 - DNA, 82 bp, Artificial; identical to SEQ ID NO: 65
  synthetic primer / aptamer

SEQ ID NO: 99 - DNA, 80 bp, Artificial
  synthetic primer / aptamer
gagacaagaataaacgctcaattattcacaatctgtcgagctagtttaactagccaagtcgttcgacaggaggctcacaa

SEQ ID NO: 100 - DNA, 82 bp, Artificial; identical to SEQ ID NO: 65
  synthetic primer / aptamer

SEQ ID NO: 101 - DNA, 82 bp, Artificial
  synthetic primer / aptamer
gggagacaagaataaacgctcaattattcacaatctgtcgagctagtttaactagccaagtcgttngacaggaggctcacaa

SEQ ID NO: 102 - DNA, 82 bp, Artificial; identical to SEQ ID NO: 68
  synthetic primer / aptamer

SEQ ID NO: 103 - DNA, 82 bp, Artificial
  synthetic primer / aptamer
gggagncaagaataaacgctcaattattcacnatctgtcgagctagtttaactagccaagtcgttcgacaggaggctcacaa

SEQ ID NO: 104 - DNA, 82 bp, Artificial
  synthetic primer / aptamer
gggagacaagaataaacgctcaattattcacgatctgtcgagctagtttaactaaccaagtcgttcgacaggaggctcacaa

SEQ ID NO: 105 - DNA, 82 bp, Artificial
  synthetic primer / aptamer
gggagacaagaataaacgcccaattattcacaatctgtcgagctagttcaactagccaagtcgttcgacaggaggctcacaa

SEQ ID NO: 106 - DNA, 82 bp, Artificial
  synthetic primer / aptamer
gggagacaagaataaacgctcaattattcacaatctgtcgagctagtttagctagccaagtcgttcgacaggaggctcacaa

SEQ ID NO: 107 - DNA, 82 bp, Artificial
  synthetic primer / aptamer
gggagacaagaataaacgctcaattattcacgatctgtcgagctagttcaactagccaagtcgttcgacaggaggctcacaa

SEQ ID NO: 108 - DNA, 82 bp, Artificial
  synthetic primer / aptamer
gggagacaagaataaacgctcaattattcacggtctgtcgagctagtttaactagccaagtcgttcgacaggaggctcacaa

SEQ ID NO: 109 - DNA, 82 bp, Artificial
  synthetic primer / aptamer
gggagacaagaataaacgctcagttattcacaatctgtcgagctagtttaactagccaagtcgttcgacaggaggctcacaa

SEQ ID NO: 110 - DNA, 82 bp, Artificial
  synthetic primer / aptamer
gggagacaagaataaacgctcaattattcacgatctgtcgagctagtttaagtagccaagtcgttcgacaggaggctcacaa

SEQ ID NO: 111 - DNA, 80 bp, Artificial
  synthetic primer / aptamer
gggagacaagaataaacgctcaattactcaggatctgtcgagctagtttaactagccaagtcgttcgacaggaggctcac

SEQ ID NO: 112 - DNA, 82 bp, Artificial
  synthetic primer / aptamer
gggagacaagaataaacgctcaattattcacaatctgtcgagntngtttatctaaccaagtngttcgacaggaggctcacaa

SEQ ID NO: 113 - DNA, 82 bp, Artificial
  synthetic primer / aptamer
gggagacaagaatgaacgctcaaagggcggtccatagtcgagtcagaataactgaccaagtcgttcgacaggaggctcacaa

SEQ ID NO: 114 - DNA, 82 bp, Artificial
  synthetic primer / aptamer
gggagacaagaagaaacgctcaaagggcggtccatagtcgagtcggaataactgaccaagtcgttcgacaggaggctcacaa

SEQ ID NO: 115 - DNA, 82 bp, Artificial
  synthetic primer / aptamer
gggagacaagaataaacgctcaaagggcggtccatagtcgagtcagaattactgaccaagtcgttcgacaggaggctcacaa

SEQ ID NO: 116 - DNA, 82 bp, Artificial
  synthetic primer / aptamer
gggagacaagaataaacgctcaagccatccaagggtcgagtaacataaatagttaccaagtcgttcgacaggaggctcacaa

SEQ ID NO: 117 - DNA, 82 bp, Artificial; identical to SEQ ID NO: 92
  synthetic primer / aptamer

SEQ ID NO: 118 - DNA, 82 bp, Artificial
  synthetic primer / aptamer
gggagacacgaataaacgctcaaaagagtaaatgtcgatcctatttatacataggacaagtcgttcgacaggaggctcacaa

SEQ ID NO: 119 - DNA, 77 bp, Artificial
  synthetic primer / aptamer
gggagacaagantaancgctcaatgaaatccagacgttggacactacggcggcagttattcgacaggaggctcacaa

SEQ ID NO: 120 - DNA, 82 bp, Artificial
  synthetic primer / aptamer
gggagacaagaataaacgctcaagtctgcagtgacctggctgatgataagtcgaatgcaaggcttcgacaggaggctcacaa

SEQ ID NO: 121 - DNA, 82 bp, Artificial
  synthetic primer / aptamer
gggaganaagaataaacgctcaantntgcagtgtcntgggagatgntnagtcgaatnnannnnttcgacaggaggctcacaa

SEQ ID NO: 122 - DNA, 82 bp, Artificial
  synthetic primer / aptamer
gggggaccagaataaacgctcaagtttgcagtgacctggctgatgataagtngaatgtaaggcttcgacaggaggctcacaa

SEQ ID NO: 123 - DNA, 82 bp, Artificial
  synthetic primer / aptamer
gggagacaagaataaacgctcaagcgagtcacatcgtgaccaagtcgttcgtaaagtgtgttattcgacaggaggctcacaa

SEQ ID NO: 124 - DNA, 82 bp, Artificial
  synthetic primer / aptamer
gggagacaagaataaacgctcaagtctgtagtgacctggctgatgataagtcgaatgtaaggcttcgacaggaggctcacaa

SEQ ID NO: 125 - DNA, 81 bp, Artificial
  synthetic primer / aptamer
gggagacaagaataaacgctcaaggcccacccttgtaaaaacaagcccaagtctggcttttattcgacaggaggctcacaa

SEQ ID NO: 126 - DNA, 82 bp, Artificial
  synthetic primer / aptamer
gggagacaagaataaacgctcaatacccagagcgtcaatggcaacagattgccagtatgtttattcgacaggaggctcacaa

SEQ ID NO: 127 - DNA, 70 bp, Artificial; identical to SEQ ID NO: 9
  synthetic primer / aptamer

SEQ ID NO: 128 - DNA, 82 bp, Artificial
  synthetic primer / aptamer
gggagacaagaataaacgctcaagcccaagataccttctctaggtatcccacccttaggcttattcgacaggaggctcacaa

SEQ ID NO: 129 - DNA, 82 bp, Artificial; identical to SEQ ID NO: 86
  synthetic primer / aptamer

SEQ ID NO: 130 - DNA, 82 bp, Artificial
  synthetic primer / aptamer
gggagacaagaataaacgctcaaagggcggtccatagtcgagtcaggataactgaccaagtcgttcgacaggaggctcacaa

SEQ ID NO: 131 - DNA, 82 bp, Artificial
  synthetic primer / aptamer
gggagacaagaataaacgctcaaagggcggtccgtagtcgagtcagaataactgaccaagtcgtttgacaggaggctcacaa

SEQ ID NO: 132 - DNA, 82 bp, Artificial; identical to SEQ ID NO: 65
  synthetic primer / aptamer

SEQ ID NO: 133 - DNA, 82 bp, Artificial; identical to SEQ ID NO: 68
  synthetic primer / aptamer

SEQ ID NO: 134 - DNA, 82 bp, Artificial; identical to SEQ ID NO: 92
  synthetic primer / aptamer

SEQ ID NO: 135 - DNA, 82 bp, Artificial; identical to SEQ ID NO: 90
  synthetic primer / aptamer

SEQ ID NO: 136 - DNA, 82 bp, Artificial
  synthetic primer / aptamer
gggagacaagaataaacgctcaacgttgtggagactggctaatttatgagtcgaatatattgcttcnacaggaggctcacaa

SEQ ID NO: 137 - DNA, 77 bp, Artificial
  synthetic primer / aptamer
gggagacaagaataaacgctcaatgaaatccagacgttggacactacggcggcagttattcgacaggaggctcacaa

SEQ ID NO: 138 - DNA, 82 bp, Artificial; identical to SEQ ID NO: 89
  synthetic primer / aptamer

SEQ ID NO: 139 - DNA, 82 bp, Artificial
  synthetic primer / aptamer
cggagacaagaataaacgctcaagtctgcagtgacctggctgatgataagtcgaatgtaaggcttcgacaggaggctcacaa

SEQ ID NO: 140 - DNA, 82 bp, Artificial; identical to SEQ ID NO: 80
  synthetic primer / aptamer

SEQ ID NO: 141 - DNA, 83 bp, Artificial
  synthetic primer / aptamer
gggagacaagaataaacgctcaacataatgtgaagcttcggggaaaatatgggaaacggacgggttcgacaggaggctcacaa

SEQ ID NO: 142 - DNA, 71 bp, Artificial
  synthetic primer / aptamer
gagacaagaataaacgggggaacagcacctaatggtgccaagtcgttgttccttcgacaggaggctcacaa

SEQ ID NO: 143 - DNA, 82 bp, Artificial
  synthetic primer / aptamer
gggaggcaagaataaacgctcaagtctgcagtgacccggctgatgataagtcgaatgtaaggcttcgacaggaggctcacaa

SEQ ID NO: 144 - DNA, 71 bp, Artificial
  synthetic primer / aptamer
gggagacaagaataaacgggagcagcacctaaaggtgccaagtcgttgctccttcgacaggaggctcacaa

SEQ ID NO: 145 - DNA, 82 bp, Artificial; identical to SEQ ID NO: 96
  synthetic primer / aptamer

SEQ ID NO: 146 - DNA, 83 bp, Artificial
  synthetic primer / aptamer
gggagacaagaataaacgctcaaaatagtgagaaatgtcgatatctcgagtaaaaactcgcccattcgacaggaggctcacaa

SEQ ID NO: 147 - DNA, 82 bp, Artificial; identical to SEQ ID NO: 96
  synthetic primer / aptamer

SEQ ID NO: 148 - DNA, 82 bp, Artificial
  synthetic primer / aptamer
gggaggcaagaataaacgctcaagcccaagataccttcactaggtatcccacccttaggcttattcgacaggaggctcacaa

SEQ ID NO: 149 - DNA, 67 bp, Artificial
  synthetic primer / aptamer
gggagacaagaataaacggagcagccaccaaagtggccaagtcgttgcttcgacaggaggctcacaa

SEQ ID NO: 150 - DNA, 67 bp, Artificial; identical to SEQ ID NO: 149
  synthetic primer / aptamer

SEQ ID NO: 151 - DNA, 69 bp, Artificial
  synthetic primer / aptamer
gggagacaagaataaacgacatccacaccgaaaggtgtccattcgttttgttcgacaggaggctcacaa

SEQ ID NO: 152 - DNA, 35 bp, Artificial
  synthetic primer / aptamer
ggagcagcaccgaaaggugccaagucguugcucct

SEQ ID NO: 153 - DNA, 11 bp, Artificial
  synthetic primer / aptamer
ggagcagcacc

SEQ ID NO: 154 - DNA, 20 bp, Artificial
  synthetic primer / aptamer
ggugccaagucguugcucct

SEQ ID NO: 155 - DNA, 18 bp, Artificial
  synthetic primer / aptamer
gugccaagucguugcuct

SEQ ID NO: 156 - DNA, 17 bp, Artificial
  synthetic primer / aptamer
gugccaagucguugcut

SEQ ID NO: 157 - DNA, 17 bp, Artificial
  synthetic primer / aptamer
ugccaagucguugcuct

SEQ ID NO: 158 - DNA, 16 bp, Artificial
  synthetic primer / aptamer
ugccaagucguugcut

SEQ ID NO: 159 - DNA, 11 bp, Artificial
  synthetic primer / aptamer
ggagcagcacc

SEQ ID NO: 160 - DNA, 20 bp, Artificial
  synthetic primer / aptamer
ggtgccaagucgutgctcct

SEQ ID NO: 161 - DNA, 18 bp, Artificial
  synthetic primer / aptamer
gtgccaagucgutgctct

SEQ ID NO: 162 - DNA, 16 bp, Artificial
  synthetic primer / aptamer
gtgccaagucgutgct

SEQ ID NO: 163 - DNA, 17 bp, Artificial
  synthetic primer / aptamer
tgccaagucgutgctct

SEQ ID NO: 164 - DNA, 15 bp, Artificial
  synthetic primer / aptamer
tgccaagucgutgct

SEQ ID NO: 165 - DNA, 20 bp, Artificial
  synthetic primer / aptamer
ggtgccaagtcgttgctcct

SEQ ID NO: 166 - DNA, 11 bp, Artificial
  synthetic primer / aptamer
ggcgcagcgcc

SEQ ID NO: 167 - DNA, 20 bp, Artificial
  synthetic primer / aptamer
ggcgccaagucguugcgcct

SEQ ID NO: 168 - DNA, 18 bp, Artificial
  synthetic primer / aptamer
gcgccaagucguugcgct

SEQ ID NO: 169 - DNA, 17 bp, Artificial
  synthetic primer / aptamer
gcgccaagucguugcgt

SEQ ID NO: 170 - DNA, 20 bp, Artificial
  synthetic primer / aptamer
ggcgccaagucguugcgcct

SEQ ID NO: 171 - DNA, 20 bp, Artificial
  synthetic primer / aptamer
ggcgccaagucguugcgcct

SEQ ID NO: 172 - DNA, 20 bp, Artificial
  synthetic primer / aptamer
ggcgccaagucguugcgcct

SEQ ID NO: 173 - DNA, 11 bp, Artificial
  synthetic primer / aptamer
ggcgcagcgcc

SEQ ID NO: 174 - DNA, 20 bp, Artificial
  synthetic primer / aptamer
ggcgccaagucguugcgcct

SEQ ID NO: 175 - DNA, 18 bp, Artificial
  synthetic primer / aptamer
gcgccaagucguugcgct

SEQ ID NO: 176 - DNA, 17 bp, Artificial
  synthetic primer / aptamer
gcgccaagucguugcgt

SEQ ID NO: 177 - DNA, 11 bp, Artificial
  synthetic primer / aptamer
ggcgcagcgcc

SEQ ID NO: 178 - DNA, 20 bp, Artificial
  synthetic primer / aptamer
ggcgccaagucguugcgcct

SEQ ID NO: 179 - DNA, 20 bp, Artificial
  synthetic primer / aptamer
ggcgccaagucguugcgcct

SEQ ID NO: 180 - DNA, 18 bp, Artificial
  synthetic primer / aptamer
gggccaagucguugccct

SEQ ID NO: 181 - DNA, 17 bp, Artificial
  synthetic primer / aptamer
gggccaagucguugcct

SEQ ID NO: 182 - DNA, 17 bp, Artificial
  synthetic primer / aptamer
ggccaagucguugccct

SEQ ID NO: 183 - DNA, 16 bp, Artificial
  synthetic primer / aptamer
ggccaagucguugcct

SEQ ID NO: 184 - DNA, 16 bp, Artificial
  synthetic primer / aptamer
gggccaagucgugcct

SEQ ID NO: 185 - DNA, 15 bp, Artificial
  synthetic primer / aptamer
ggccaagucgugcct

SEQ ID NO: 186 - DNA, 11 bp, Artificial
  synthetic primer / aptamer
ggagcagcacc

SEQ ID NO: 187 - DNA, 20 bp, Artificial
  synthetic primer / aptamer
ggugccaagucguugcucct

SEQ ID NO: 188 - DNA, 20 bp, Artificial
  synthetic primer / aptamer
ggugccaagucguugcucct

SEQ ID NO: 189 - DNA, 20 bp, Artificial
  synthetic primer / aptamer
ggugccaagucguugcucct

SEQ ID NO: 190 - DNA, 20 bp, Artificial
  synthetic primer / aptamer
ggugccaagucguugcucct

SEQ ID NO: 191 - DNA, 20 bp, Artificial
  synthetic primer / aptamer
ggugccaagucguugcucct

SEQ ID NO: 192 - DNA, 20 bp, Artificial
  synthetic primer / aptamer
ggugccaagucguugcucct

SEQ ID NO: 193 - DNA, 20 bp, Artificial
  synthetic primer / aptamer
ggugccaagucguugcucct

SEQ ID NO: 194 - DNA, 20 bp, Artificial
  synthetic primer / aptamer
ggugccaagucguugcucct

SEQ ID NO: 195 - DNA, 11 bp, Artificial
  synthetic primer / aptamer
ggagcagcacc

SEQ ID NO: 196 - DNA, 11 bp, Artificial
  synthetic primer / aptamer
ggagcagcacc

SEQ ID NO: 197 - DNA, 11 bp, Artificial
  synthetic primer / aptamer
ggagcagcacc

SEQ ID NO: 198 - DNA, 11 bp, Artificial
  synthetic primer / aptamer
ggagcagcacc

SEQ ID NO: 199 - DNA, 11 bp, Artificial
  synthetic primer / aptamer
ggagcagcacc

SEQ ID NO: 200 - DNA, 11 bp, Artificial
  synthetic primer / aptamer
ggagcagcacc

SEQ ID NO: 201 - DNA, 20 bp, Artificial
  synthetic primer / aptamer
ggugccaagucguugcucct

SEQ ID NO: 202 - DNA, 11 bp, Artificial
  synthetic primer / aptamer
ggagcagcacc

SEQ ID NO: 203 - DNA, 11 bp, Artificial
  synthetic primer / aptamer
ggagcagcacc

SEQ ID NO: 204 - DNA, 11 bp, Artificial
  synthetic primer / aptamer
ggagcagcacc

SEQ ID NO: 205 - DNA, 11 bp, Artificial
  synthetic primer / aptamer
ggagcagcacc

SEQ ID NO: 206 - DNA, 20 bp, Artificial
  synthetic primer / aptamer
ggugccaagucguugcucct

SEQ ID NO: 207 - DNA, 20 bp, Artificial
  synthetic primer / aptamer
ggugccaagucguugcucct

SEQ ID NO: 208 - DNA, 11 bp, Artificial
  synthetic primer / aptamer
ggagcagcacc

SEQ ID NO: 209 - DNA, 20 bp, Artificial
  synthetic primer / aptamer
ggugccaagucguugcucct

SEQ ID NO: 210 - DNA, 11 bp, Artificial
  synthetic primer / aptamer
ggagcagcacc

SEQ ID NO: 211 - DNA, 11 bp, Artificial
  synthetic primer / aptamer
ggagcagcacc

SEQ ID NO: 212 - DNA, 20 bp, Artificial
  synthetic primer / aptamer
ggugccaagucguugcucct

SEQ ID NO: 213 - DNA, 20 bp, Artificial
  synthetic primer / aptamer
ggugccaagucguugcucct

SEQ ID NO: 214 - DNA, 20 bp, Artificial
  synthetic primer / aptamer
ggugccaagucguugcucct

SEQ ID NO: 215 - DNA, 11 bp, Artificial
  synthetic primer / aptamer
ggagcagcacc

SEQ ID NO: 216 - DNA, 20 bp, Artificial
  synthetic primer / aptamer
ggugccaagucguugcucct

SEQ ID NO: 217 - DNA, 20 bp, Artificial
  synthetic primer / aptamer
ggugccaagucguugcucct

SEQ ID NO: 218 - DNA, 20 bp, Artificial
  synthetic primer / aptamer
ggugccaagucguugcucct

SEQ ID NO: 219 - DNA, 20 bp, Artificial
  synthetic primer / aptamer
ggugccaagucguugcucct

SEQ ID NO: 220 - DNA, 20 bp, Artificial
  synthetic primer / aptamer
ggugccaagucguugcucct

SEQ ID NO: 221 - DNA, 20 bp, Artificial
  synthetic primer / aptamer
ggugccaagtcguugcucct

SEQ ID NO: 222 - DNA, 20 bp, Artificial
  synthetic primer / aptamer
ggugccaagucguugcucct

SEQ ID NO: 223 - DNA, 20 bp, Artificial
  synthetic primer / aptamer
ggugccaagucguugcucct

SEQ ID NO: 224 - DNA, 20 bp, Artificial
  synthetic primer / aptamer
ggugccaagucgtugcucct

SEQ ID NO: 225 - DNA, 20 bp, Artificial
  synthetic primer / aptamer
ggugccaagtcgtugcucct

SEQ ID NO: 226 - DNA, 16 bp, Artificial
  synthetic primer / aptamer
gggagaggagagaacg

SEQ ID NO: 227 - DNA, 23 bp, Artificial
  synthetic primer / aptamer
cggctagtcagtcgcgatgcatg

SEQ ID NO: 228 - DNA, 33 bp, Artificial
  synthetic primer / aptamer
taatacgactcactatagggagaggagagaacg

SEQ ID NO: 229 - DNA, 23 bp, Artificial
  synthetic primer / aptamer
catgcatcgcgactgactagccg

SEQ ID NO: 230 - DNA, 17 bp, Artificial
  synthetic primer / aptamer
ggagcgcactcagccac

SEQ ID NO: 231 - DNA, 19 bp, Artificial
  synthetic primer / aptamer
tttcgacctctctgctagc

SEQ ID NO: 232 - DNA, 34 bp, Artificial
  synthetic primer / aptamer
taatacgactcactataggagcgcactcagccac

SEQ ID NO: 233 - DNA, 19 bp, Artificial
  synthetic primer / aptamer
gctagcagagaggtcgaaa

SEQ ID NO: 234 - protein, 13 aa, Human immunodeficiency virus
QNQQEKNEQELLE

SEQ ID NO: 235 - protein, 10 aa, Human immunodeficiency virus
DKWDSLWNWF

SEQ ID NO: 236 - protein, 24 aa, Human immunodeficiency virus
QNQQEKNEQELLELDKWASLWNWF

SEQ ID NO: 237 - protein, 36 aa, Human immunodeficiency virus
YTSLIHSLIEESQNQQEKNEQELLELDKWASLWNWF

SEQ ID NO: 238 - protein, 34 aa, Human immunodeficiency virus
WMEWDREINNYTSLIHSLIEESQNQQEKNEQELL

SEQ ID NO: 239 - DNA, 9 bp, Artificial
  synthetic primer / aptamer
gagcagcac

SEQ ID NO: 240 - DNA, 8 bp, Artificial
  synthetic primer / aptamer
agcagcac

SEQ ID NO: 241 - DNA, 8 bp, Artificial
  synthetic primer / aptamer
gagcagca

SEQ ID NO: 242 - DNA, 7 bp, Artificial
  synthetic primer / aptamer
agcagca

SEQ ID NO: 243 - DNA, 9 bp, Artificial
  synthetic primer / aptamer
gcgcagcgc

SEQ ID NO: 244 - DNA, 8 bp, Artificial
  synthetic primer / aptamer
cgcagcgc

SEQ ID NO: 245 - DNA, 9 bp, Artificial
  synthetic primer / aptamer
gggcagccc

SEQ ID NO: 246 - DNA, 8 bp, Artificial
  synthetic primer / aptamer
ggcagccc

SEQ ID NO: 247 - DNA, 8 bp, Artificial
  synthetic primer / aptamer
gggcagcc

SEQ ID NO: 248 - DNA, 7 bp, Artificial
  synthetic primer / aptamer
ggcagcc

SEQ ID NO: 249 - DNA, 7 bp, Artificial
  synthetic primer / aptamer
ggcgccc

SEQ ID NO: 250 - DNA, 6 bp, Artificial
  synthetic primer / aptamer
ggcgcc